(19) United States
(12) Patent Application Publication
(10) Pub. No.: US 2022/0333089 A1
(43) Pub. Date: Oct. 20, 2022

(54) Split CPF1 Protein

(71) Applicant: The University of Tokyo, Tokyo (JP)

(72) Inventors: Moritoshi Sato, Tokyo (JP); Takahiro Otabe, Tokyo (JP); Yuta Nihongaki, Tokyo (JP)

(21) Appl. No.: 17/290,317

(22) PCT Filed: Nov. 1, 2019
(86) PCT No.: PCT/JP2019/043161

§ 371 Date: Jun. 13, 2022

(30) Foreign Application Priority Data
Nov. 1, 2018 (JP) 2018-206940

(51) Int. Cl. C12N 9/22; C12N 15/63; C12N 15/11

(52) U.S. Cl. CPC C12N 9/22; C12N 15/63; C12N 15/111; C12N 2310/20

(57) ABSTRACT

The present invention provides a set of two polypeptides of a split Cpf1 protein, wherein the two polypeptides are a N-terminal side fragment of a Cpf1 protein and a C-terminal side fragment of the Cpf1 protein.

### TECHNICAL FIELD

The present invention relates to a split Cpf1 protein.

### BACKGROUND ART

In recent years, as a genome editing tool which can cut a desired target DNA sequence in a genome, the CRISPR (clustered regularly interspaced palindromic repeats)-Cas9 system has been developed (Non-Patent Documents 1-3). In this system, a guide RNA which guides Streptococcus pyogenes-derived Cas9 nuclease Cas9) and Cas9 to a target DNA sequence is used. A PAM (protospacer-adjacent motif) region which is complementary to first 20 bases of the guide RNA and is represented by NGG (N represents any base of A, T, C and G) of a C-terminal side thereof, becomes a target DNA sequence, and is cut with Cas9.

The CRISPR-Cas9 system is a powerful tool which can simply and precisely cut an arbitrary sequence by designing an appropriate guide RNA, and can perform genome editing by introducing an arbitrary indel mutation (insertion/deletion mutation) into a cutting site when non-homologous end-joining (NHEJ) and homology-directed repair (HDR) are combined.

In addition, various modification techniques for genome editing are known, the techniques using fused proteins of nuclease-inactive mutated Cas9 (dead Cas9: dCas9) or nickase-type mutated Cas9 (Cas9 nickase: nCas9) and various effectors.

Meanwhile, a molecule controlling approach utilizing photoactivation of a protein has appeared, and is called optogenetics (Non-Patent Documents 4, 5). The present inventors altered the Neurospora Crassa-derived Vivid protein which forms a homodimer with dependence on light, and developed a pair of light switch proteins “Magnet”, which can precisely control formation and dissociation of a dimer by irradiation of light (Non-Patent Document 6). In addition, the present inventors developed a set of two fused polypeptides obtained by fusing a split Cas9 protein and a Magnet as a genome editing tool (Non-Patent Document 7, Patent Document 2).

In recent years, Francisella tularensis-derived Cpf1 nuclease (Cpf1) has been discovered as a Class 2 endonuclease of the CRISPR-Cas9 system, and utilized as a genome editing tool (Non-Patent Document 8, Patent Documents 3, 4).

For Cpf1, crRNA which guides it to a target DNA sequence is used. A PAM (protospacer-adjacent motif) region which is complementary to 20 to 25 bases from the 3′-terminus of crRNA and whose 5′-terminal side is represented by TTTV (V represents any base of A, C and G) becomes a target DNA sequence, and is cut with Cpf1.

### CITATION LIST

### Patent Documents

Patent Document 1 JP2015-165776A1

Patent Document 2 WO2016/167300

Patent Document 3 US2016/208243A1

Patent Document 4 WO2017/106657A1

### Non-Patent Documents

Non-Patent Document 1 Cong, L. et al., Science 339, 819-823 (2013).

Non-Patent Document 2 Mali, P. et al., Science 339, 823-826 (2013).

Non-Patent Document 3 Jinek, M. et al., Elife 2, e00471 (2013).

Non-Patent Document 4 Toettcher, J. E. et al., Nat. Methods 8, 35-38 (2011).

Non-Patent Document 5 Mueller, K. et al., Mol. BioSyst. 9, 596-608 (2013).

Non-Patent Document 6 Kawano, F. et al., Nat. Commun. 6, 6256 (2015).

Non-Patent Document 7 Nihongaki Y. et al., Nat. Biotech, 33, 755 (2015) .

Non-Patent Document 8 Zetsche, B. et al., Cell, 163, 1 (2015).

### SUMMARY

### Technical Problem

A technical problem of the present invention is to provide a novel genome editing technique using a Cpf1 protein.

### Solution to Problem

In order to solve the problem, the present inventors made fragments obtained by dividing a Cpf1 protein into two at a variety of positions, and found out that the split Cpf1 protein is rearranged through induced association or spontaneous association.

Based on these findings, we completed the present invention.

That is, the present invention is as follows:

[1]

A set of two polypeptides of a split Cpf1 protein, wherein the two polypeptides are a N-terminal side fragment of a Cpf1 protein and a C-terminal side fragment of the Cpf1 protein.

[2]

The set of polypeptides according to [1], wherein the set is a set of two fused polypeptides of a split Cpf1 protein in which one of the N-terminal side fragment and the C-terminal side fragment of the Cpf1 protein is bound to each of two polypeptides which form a dimer with dependence on light or in the presence of a drug.

[3]

The set of polypeptides according to [1] or [2], wherein the N-terminal side fragment of the Cpf1 protein and the C-terminal side fragment of the Cpf1 protein spontaneously associate.

[4]

The set of polypeptides according to any one of [1] to [3], wherein the Cpf1 protein is nuclease-active.

[5]

The set of polypeptides according to any one of [1] to [3], wherein the Cpf1 protein is nuclease-inactive.

[6]

The set of polypeptides according to [5], wherein a functional domain is bound to the N-terminal side fragment of the Cpf1 protein and/or the C-terminal side fragment of the Cpf1 protein.

[7]

The set of polypeptides according to [1],

wherein the Cpf1 protein is nuclease-inactive,

the N-terminal side fragment of the Cpf1 protein and the C-terminal side fragment of the Cpf1 protein spontaneously associate, and

the N-terminal side fragment of the Cpf1 protein and/or the C-terminal side fragment of the Cpf1 protein is/are bound to one of two polypeptides which form a dimer with dependence on light or in the presence of a drug, and a functional domain is bound to the other of the two polypeptides which form a dimer with dependence on light or in the presence of a drug.

[8]

The set of polypeptides according to any one of [1] to [7],

wherein the N-terminal side fragment of the Cpf1 protein and the C-terminal side fragment of the Cpf1 protein are any of the following:

any of combinations of two polypeptides obtained by cutting an amino acid sequence of SEQ ID No.: 2 at any position of position 69 to position 73, position 83 to position 89, position 131 to position 138, position 244 to position 252, position 265 to position 296, position 309 to position 312, position 371 to position 387, position 404 to position 409, position 437 to position 445, position 549 to position 552, position 567 to position 577, position 606 to position 609, position 619 to position 628, position 727 to position 736, position 802 to position 811, position 1037 to position 1042, position 1140 to position 1148, position 1155 to position 1161, and position 1163 to position 1178;

a combination such that a sequence of at least one fragment in any of the above combinations includes addition, substitution, or deletion of one to several amino acids; and a combination such that a sequence of at least one fragment in any of the above combinations has 80% or more sequence identity with the above sequence.

[9]

A nucleic acid encoding the set of polypeptides according to any one of [1] to [8].

[10]

An expression vector comprising the nucleic acid according to [9].

[11]

A method of cutting a target double-stranded nucleic acid, the method including:

a step of incubating the target double-stranded nucleic acid, the set of polypeptides according to [4].

[12]

A method of cutting a target double-stranded nucleic acid, the method including:

a step of incubating the target double-stranded nucleic acid, the set of polypeptides according to [4], and a pair of guide RNAs including a sequence complementary to each sequence of the target double-stranded nucleic acid, by irradiating light, or in the presence of a drug.

[13]

A method of suppressing or activating expression of a target gene, the method including:

a step of incubating the target gene and the set of polypeptides according to [6].

[14]

A method of suppressing or activating expression of a target gene, the method including:

a step of incubating the target gene, the set of polypeptides according to [6], and a pair of guide RNAs including a sequence complementary to each sequence of the target double-stranded nucleic acid, by irradiating light, or in the presence of a drug.

[15]

A method of suppressing or activating expression of a target gene, the method including:

a step of incubating the target gene and the set of polypeptides according to [7], by irradiating light, or in the presence of a drug.

### Advantageous Effects of Invention

According to the present invention, a novel genome editing technique using a Cpf1 protein can be provided.

### BRIEF DESCRIPTION OF DRAWINGS

FIG. 1 exhibits an outline of a bioluminescence assay system for evaluation of the DNA cutting efficiency (genome editing efficiency) of split Cpf1 (split-Cpf1). An N-terminal side fragment of Cpf1 (split-Cpf1-N) and a C-terminal side fragment of Cpf1 (split-Cpf1-C) prepared by dividing Cpf1 into two are respectively ligated with FRB and FKBP, which form a dimer by addition of rapamycin. HEK293T cells are transfected with plasmids encoding the two fused proteins (split-Cpf1-N-FRB and FKBP-split-Cpf1-C) and a plasmid encoding a guide RNA (crRNA), and the fused proteins and the crRNA are expressed. To evaluate the DNA cutting efficiency of split-Cpf1 prepared as described, a bioluminescence assay system is constructed. This assay system uses an expression vector for luciferase with a stop codon introduced in the middle thereof (StopFluc reporter; using pCMV as a promoter) and a vector for luciferase without a promoter (Fluc donor). When the StopFluc reporter is cut (double strand break; DSB) by split-Cpf1, repair based on homologous recombination (homology directed repair; HDR) with the Fluc donor occurs, allowing luciferase to be expressed. The DNA cutting efficiency of split-Cpf1 is evaluated by measuring the bioluminescence signal of luciferase. Split-Cpf1 was prepared by dividing Cpf1 into two at a variety of positions, and the DNA cutting efficiency thereof was evaluated for the case where rapamycin was added to allow a dimer of FKBP and FRB to form and the case where rapamycin was not added not to allow a dimer of FKBP and FRB to form.

FIG. 2 exhibits a schematic diagram of genome editing by split-Cpf1 using an FKBP-rapamycin-FRB system with FKBP and FRB as drug switch proteins and rapamycin as a drug which induces dimer formation of FKBP and FRB. FKBP and FRB are proteins which form a dimer by addition of rapamycin, and the two fused proteins (split-Cpf1-N-FRB and FKBP-split-Cpf1-C) undergo the association of split-Cpf1-N-FRB and FKBP-split-Cpf1-C through formation of a dimer of FKBP and FRB by addition of rapamycin, and split-Cpf1 is rearranged.

FIG. 3 exhibits the results of comparison of difference in DNA cutting efficiency (genome editing efficiency) among different dividing positions for split-Cpf1 by using split-Cpf1 with the FKBP-rapamycin-FRB system (split-Cpf1-N-FRB and FKBP-split-Cpf1-C) in the presence of rapamycin (Rapamycin(+)) and in the absence thereof (Rapamycin(−)). For split-Cpf1 obtained by using the bioluminescence assay system in FIG. 1 and dividing Cpf1 derived from the Lachnospiraceae bacterium ND2006 (LbCpf1) at a variety of positions (e.g., “N70/C71” in the figure is split-Cpf1 prepared by dividing between the 70th amino acid residue and the 71st amino acid residue), the DNA cutting efficiencies were compared between the case where rapamycin was added to allow FKBP and FRB to form a dimer and the case where rapamycin was not added not to allow FKBP and FRB to form a dimer (the data in the figure had been normalized with respect to a bioluminescence signal provided by full length LbCpf1 (indicated as “Full length”, also referred to as “full length LbCpf1”) in the absence of rapamycin). The result found split-Cpf1 which exhibited the increase in DNA cutting efficiency with dependence on addition of rapamycin, that is, dimer formation of FKBP and FRB ligated to split-Cpf1. Additionally discovered was split-Cpf1 which exhibited high DNA cutting efficiency even without addition of rapamycin, that is, without induction of dimer formation of FKBP and FRB ligated. The former is “inducibly associative split-Cpf1”, the DNA cutting activity (genome editing activity) of which can be controlled through the rearrangement of split-Cpf1 caused by drug-induced external stimulation, and the latter is “spontaneously associative split-Cpf1”, the DNA cutting activity (genome editing activity) of which is evoked by the rearrangement of split-Cpf1 caused by spontaneous association irrespective of external stimulation. The subsequent evaluations were performed for N730/C731 (right arrow) as inducibly associative split-Cpf1 and N574/C575 (left arrow) as spontaneously associative split-Cpf1. The spontaneously associative split-Cpf1 of N574/C575 exhibited much higher activity than the full length Cpf1. The inducibly associative split-Cpf1 of N730/C731 exhibited low activity in the absence of rapamycin, but exhibited high inducibility in the presence of rapamycin, thus being split-Cpf1 having high selectivity as drug-inducibly associative type.

FIG. 4 exhibits the result of evaluation of genome editing by drug-inducibly associative split-Cpf1 (N730/C731) from LbCpf1 using a drug switch protein (FRB-rapamycin-FKBP system) (cells: HEK293T cells; target site of genome: DNMT1 (site 1); comparison with full length LbCpf1 (Cpf1)).

FIG. 5 exhibits the result of evaluation of genome editing by light-inducibly associative split-Cpf1 (N730/C731) from LbCpf1 using a light switch protein (pMag-nMagHigh1 system) (cells: HEK293T cells; target site of genome: DNMT1 (site 1); comparison with full length LbCpf1 (Cpf1)).

FIG. 6 exhibits the result of evaluation of genome editing by light-inducibly associative split-Cpf1 (N730/C731) from LbCpf1 using a light switch protein (pMag-nMagHigh1 system) (cells: HEK293T cells; target site of genome: GRIN2b, FANCF (site 1), FANCF (site 2); comparison between light-inducibly associative split-Cpf1 (paCpf1) and full length LbCpf1 (Cpf1); D: without light irradiation; L: with light irradiation).

FIG. 7 exhibits the result of evaluation of genome editing by light-inducibly associative split-Cpf1 (N730/C731) from LbCpf1 using a light switch protein (pMag-nMagHigh1 system) (cells: HeLa cells; target site of genome: DNMT1 (site 1), VEGFA; comparison between light-inducibly associative split-Cpf1 (paCpf1) and full length LbCpf1 (Cpf1)).

FIG. 8 exhibits the result of evaluation of genome editing by light-inducibly associative split-Cpf1 (N730/C731) from LbCpf1 using a light switch protein (pMag-nMagHigh1 system) (cells: HeLa cells; target site of genome: GRIN2b, FANCF (site 1); comparison between light-inducibly associative split-Cpf1 (paCpf1) and full length LbCpf1 (Cpf1 )).

FIG. 9 exhibits the result of spatial control of genome editing by light-inducibly associative split-Cpf1 (N730/C731) from LbCpf1 using a light switch protein (pMag-nMagHigh1 system). Spatial control of genome editing was evaluated by using a surrogate EGFP reporter.

FIG. 10 exhibits the result of transcription activation by drug-inducibly associative split-dCpf1 from LbCpf1. A drug switch protein (FRB-rapamycin-FKBP system) and a transcription activation domain (VPR) were ligated to drug-inducibly associative split-dCpf1 (dN730/dC731; dC731 is a C-terminal side fragment of dCpf1, which is obtained by introducing E925A mutation into a C-terminal side fragment of split-Cpf1 (N730/C731) to delete the nuclease activity, and dN730 is an N-terminal side fragment of the split-dCpf1; likewise, other types of split-dCpf1 have the mutation of E925A), and the drug-induced transcription activity was evaluated. An FRB-rapamycin-FKBP system was used, and a GAL4-luciferase reporter was used for evaluation of transcription activity (comparison between w/: with addition of rapamycin (left) and w/o: without addition of rapamycin (right)).

FIG. 11 exhibits the result of genome editing by spontaneously associative split-Cpf1 from LbCpf1. The spontaneously associative split-Cpf1 (N574/C575) exhibited nuclease activity both when the dimerization domains (FKBP, FRB) were ligated and rapamycin was not added (the leftmost data) and when the dimerization domains (FKBP, FRB) were not ligated (the second date from the left). When spontaneously associative split-dCpf1 (dN574/dC575; dC575 is a C-terminal side fragment of dCpf1, which is obtained by introducing E925A mutation into a C-terminal side fragment of split-Cpf1 (N574/C575) to delete the nuclease activity, and dN574 is an N-terminal side fragment of split-dCpf1) was used (the third data from the left), no nuclease activity was exhibited.

FIG. 12 exhibits the result of induction of transcription activity with a drug by ligating a drug switch protein and a transcription activation domain (p65-HSF1) to spontaneously associative split-dCpf1 (dN574/dC575) from LbCpf1. An FRB-rapamycin-FKBP system (rapamycin as the drug), a PYL-abscisic acid (ABA)-ABI system (ABA as the drug), or a GID1-GA3-AM-GAI system (GA3-AM as the drug) was used as the drug switch protein. FKBP was ligated to p65-HSF1 in ligating FRB to split-dCpf1, and FRB was ligated to p65-HSF1 in ligating FKBP to split-dCpf1. API was ligated to p65-HSF1 in ligating PYL to split-dCpf1, and PYL was ligated to p65-HSF1 in ligating API to split-dCpf1. GAI was ligated to p65-HSF1 in ligating GID1 to split-dCpf1, and GID1 was ligated to p65-HSF1 in ligating GAI to split-dCpf1. A GAL4-luciferase reporter was used for evaluation of transcription activity. For each type of split-dCpf1, comparison was made between the case with addition of a drug (right) and the case without addition of a drug (left). When p65 was ligated to each fragment of spontaneously associative split-dCpf1 (dN574/dC575) (the second data from the right), very high transcription activity was exhibited.

FIG. 13 exhibits the result of induction of the transcription activity of a genomic gene (ASCL1) with a drug by spontaneously associative split-dCpf1 (dN574/dC575) from LbCpf1.

FIG. 14 exhibits the result of induction of transcription activity with light by spontaneously associative split-dCpf1 (dN574/dC575) from LbCpf1. A CRY2-CIB1 system was used as a light switch protein. CIB1 was ligated to split-dCpf1, and CRY2-PHR was ligated to a transcription activation domain (p65-HSF1). One to four CIB1 were ligated to the four termini (two N-termini and two C-termini) present in the fragments of split-dCpf1 (dN574/dC575), and the transcription activity for each case was compared with that when one CIB1 was ligated to full length dLbCpf1 (Full length dLbCpf1). Dark indicates the case without light irradiation (left), and Light indicates the case with light irradiation (right). A GAL4-luciferase reporter was used for evaluation of transcription activity.

FIG. 15 exhibits the result of induction of the transcription activity of a genomic gene (ASCL1) with light by spontaneously associative split-dCpfl1 (dN574/dC575) from LbCpf1.

FIG. 16 exhibits the result of transcription activation by spontaneously associative split-dCpf1 (dN574/dC575) from LbCpf1. A transcription activation domain was ligated to spontaneously associative split-dCpf1, and the transcription activity caused by spontaneous association was evaluated. One to four transcription activation domains were ligated to the four termini (two N-termini and two C-termini) present in the fragments of split-dCpf1 (dN574/dC575), and the transcription activity was evaluated. VP64, VPR, and p65 were used as the transcription activation domains. A GAL4-luciferase reporter was used for evaluation of transcription activity.

FIG. 17 exhibits the result of transcription activation for a genomic gene (ASCL1) by spontaneously associative split-dCpf1 (dN574/dC575) from LbCpf1.

FIG. 18 exhibits the result of transcription activation for a genomic gene (ASCL1) by spontaneously associative split-dCpf1 from LbCpf1. Comparison between divided dCpf1 activators (BPNLS-p65-HSF1-NLS-dN574-p65-HSF1-BPNLS and BPNLS-p65-HSF1-dC575-p65-HSF1-BPNLS) and a probe in which p65-HSF1 was ligated to each of the N-terminus and the C-terminus of full length dLbCpf1 (BPNLS-p65-HSF1-dCpf1-p65-HSF1-BPNLS).

FIG. 19 exhibits the result of transcription activation for a genomic gene (MYOD1) by spontaneously associative split-dCpf1 from LbCpf1. Comparison between divided dCpf1 activators (BPNLS-p65-HSF1-NLS-dN574-p65-HSF1-BPNLS and BPNLS-p65-HSF1-dC575-p65-HSF1-BPNLS) and a probe in which p65-HSF1 was ligated to each of the N-terminus and the C-terminus of full length dLbCpf1 (BPNLS-p65-HSF1-dCpf-p65-HSF1-BPNLS).

FIG. 20 exhibits a conceptual diagram of induction of differentiation of iPS cells using transcription activation by spontaneously associative split-dCpf1 from LbCpf1. iPS cells are differentiated into nerve cells through activation of transcription of a genomic gene (Neurogenin3) using a divided dCpf1 activator.

FIG. 21 exhibits the result of induction of differentiation of iPS cells using transcription activation by spontaneously associative split-dCpf1 from LbCpf1. Transcription of a genomic gene (Neurogenin3) was activated with a divided dCpf1 activator (BPNLS-p65-HSF1-NLS-dN574-p65-HSF1-BPNLS and BPNLS-p65-HSF1-dC575-p65-HSF1-BPNLS). Comparison was made between each case with any one of six crRNAs (crNGN3-1 to 3-6) targeting Neurogenin3 and the case with a mixture of all of them (crNGN3_Mix).

FIG. 22 exhibits the result of induction of differentiation of iPS cells using transcription activation by spontaneously associative split-dCpf1 from LbCpf1. iPS cells are differentiated into nerve cells through activation of transcription of a genomic gene (Neurogenin3) using a divided dCpf1 activator (BPNLS-p65-HSF1-NLS-dN574-p65-HSF1-BPNLS and BPNLS-p65-HSF1-dC575-p65-HSF1-BPNLS). Comparison was made between each case with any one of six crRNAs (crNGN3-1 to 3-6) targeting Neurogenin3 and the case with a mixture of all of them (crNGN3_Mix).

FIG. 23 exhibits the amino acid sequence of an LpCpf1-NLS-3xHA tag containing the full length amino acid sequence of LbCpf1. In the amino acid sequence, NLS means Nucleoplasmin NLS, and is a nuclear localization sequence. In subsequent FIG. 23 to FIG. 36, each nuclear localization sequence is shown with hatching, and each switch protein which forms a dimer with dependence on light or in the presence of a drug is shown with enclosure. In FIG. 23 to FIG. 36, each underline indicates a starting amino acid (M), each double underline indicates a restriction enzyme site, and each dashed underline indicates a linker.

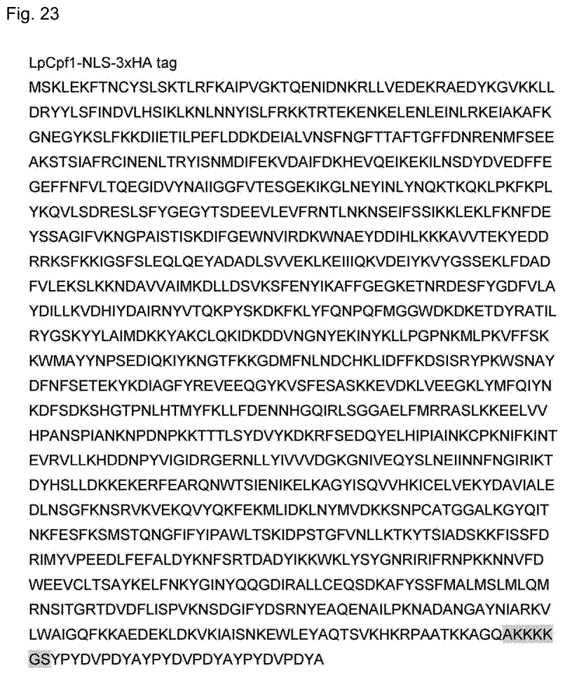

FIG. 24 exhibits the amino acid sequence of NLS-N730-FRB containing split-Cpf1-N. In the amino acid sequence, NLS means SV40 NLS, and is a nuclear localization sequence. N730 is split-Cpf1-N formed by cutting LbCpf1 at a cutting site of N730/C731. FRB is a drug switch protein which forms a dimer by addition of rapamycin.

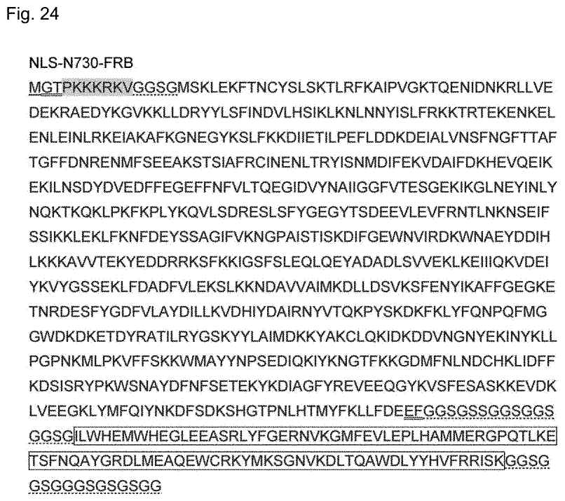

FIG. 25 exhibits the amino acid sequence of FKBP-N731-NLS containing split-Cpf1-C. In the amino acid sequence, NLS means Nucleoplasmin NLS, and is a nuclear localization sequence. C731 is split-Cpf1-C formed by cutting LbCpf1 at a cutting site of N730/C731. FKBP is a drug switch protein which forms a dimer by addition of rapamycin.

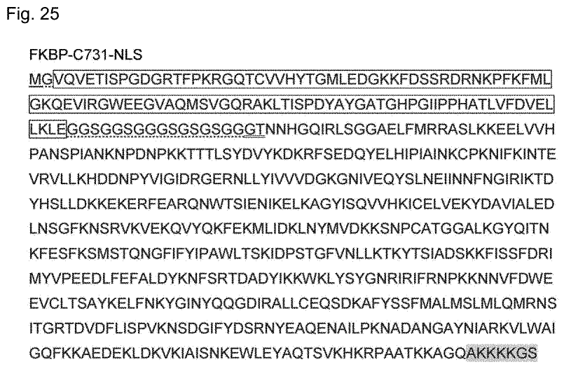

FIG. 26 exhibits the amino acid sequence of NLS-N730-pMag containing split-Cpf1-N. In the amino acid sequence, NLS means SV40 NLS, and is a nuclear localization sequence. N730 is split-Cpf1-N formed by cutting LbCpf1 at a cutting site of N730/C731. pMag is a light switch protein (pMag-nMagHigh1 system).

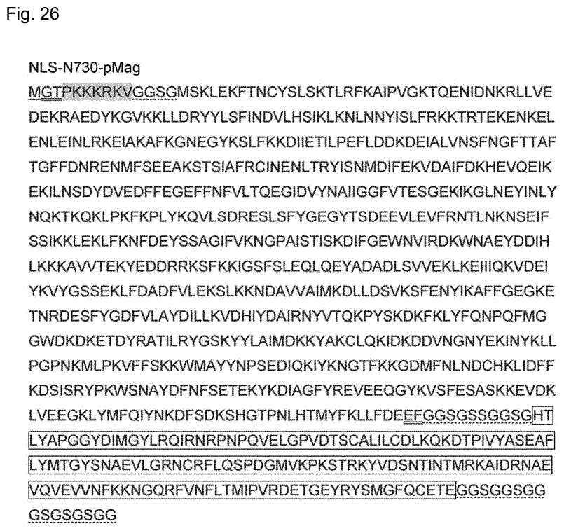

FIG. 27 exhibits the amino acid sequence of nMagHigh1-N731-NLS containing split-Cpf1-C. In the amino acid sequence, NLS means Nucleoplasmin NLS, and is a nuclear localization sequence. C731 is split-Cpf1-C formed by cutting LbCpf1 at a cutting site of N730/C731. nMagHigh1 is a light switch protein (pMag-nMagHigh1 system).

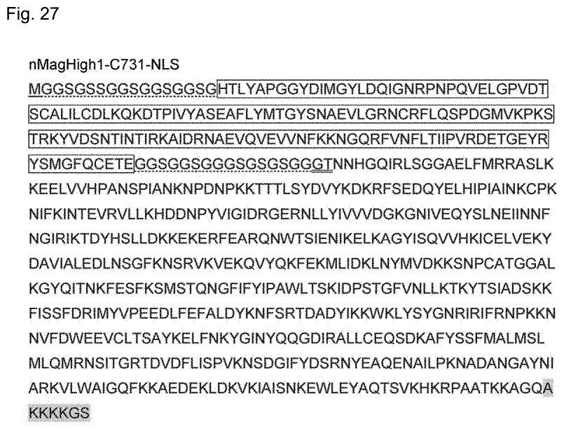

FIG. 28 exhibits the amino acid sequence of NLSx3-dN730-FRB-NLS containing split-dCpf1-N. In the amino acid sequence, NLS means SV40 NLS, and is a nuclear localization sequence, and x3 means three times repeat. dN730 is split-dCpf1-N formed by cutting dLbCpf1 at a cutting site of N730/C731. FRB is a drug switch protein which forms a dimer by addition of rapamycin.

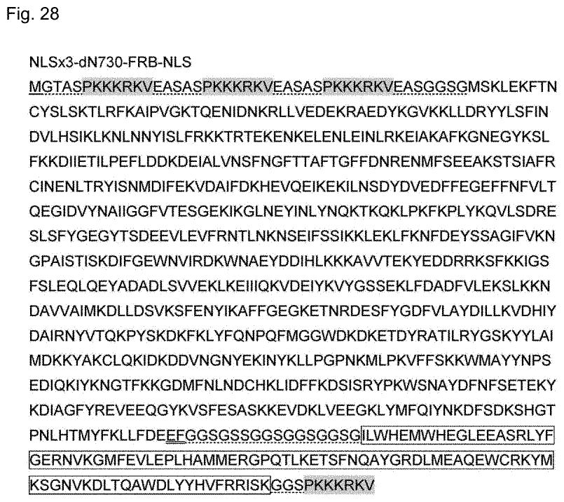

FIG. 29 exhibits the amino acid sequence of VPR-FKBP-dC731-NLS containing split-dCpf1-C. In the amino acid sequence, NLS means Nucleoplasmin NLS, and is a nuclear localization sequence. dC731 is split-dCpf1-C formed by cutting dLbCpf1 at a cutting site of N730/C731. VPR is a transcription activation domain, and FKBP is a drug switch protein which forms a dimer by addition of rapamycin. In subsequent FIG. 29 to FIG. 36, each transcription activation domain is indicated with hatching and enclosure.

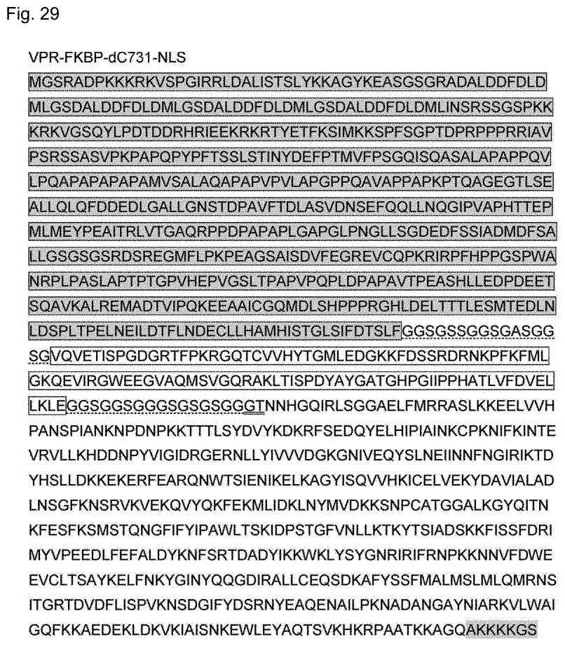

FIG. 30 exhibits the amino acid sequence of NLS-N574-NLS containing split-Cpf1-N. In the amino acid sequence, NLS means SV40 NLS, and is a nuclear localization sequence. N574 is split-Cpf1-N formed by cutting LbCpf1 at a cutting site of N574/C575.

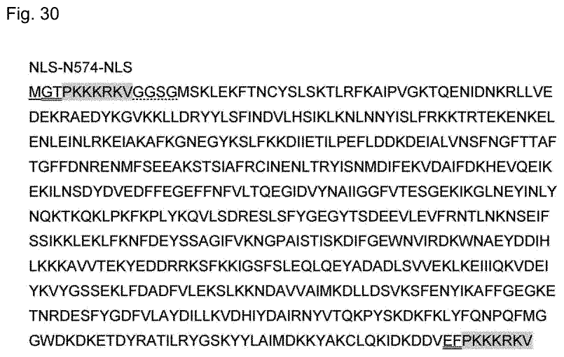

FIG. 31 exhibits the amino acid sequence of NLS-C575-NLS containing split-Cpf1-C. In the amino acid sequence, NLS in the N-terminal side means NLSV40 NLS, and NLS in the C-terminal side means Nucleoplasmin NLS, each being a nuclear localization sequence. C575 is split-Cpf1-C formed by cutting LbCpf1 at a cutting site of N574/C575.

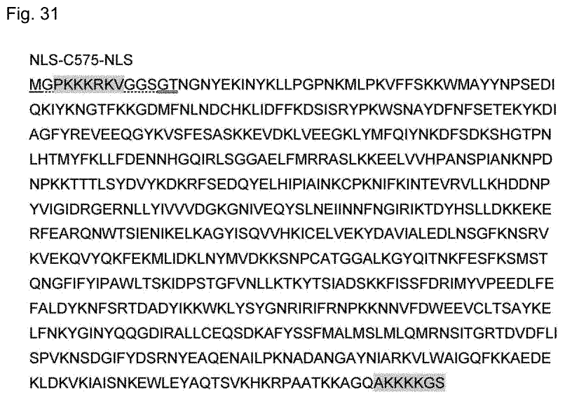

FIG. 32 exhibits the amino acid sequence of BPNLS-CIB1-dN574-CIB1-BPNLS containing split-dCpf1-N. In the amino acid sequence, BPNLS is a nuclear localization sequence. dN574 is split-dCpf1-N formed by cutting dLbCpf1 at a cutting site of N574/C575. CIB1 is a light switch protein (CRY2-CIB1 system).

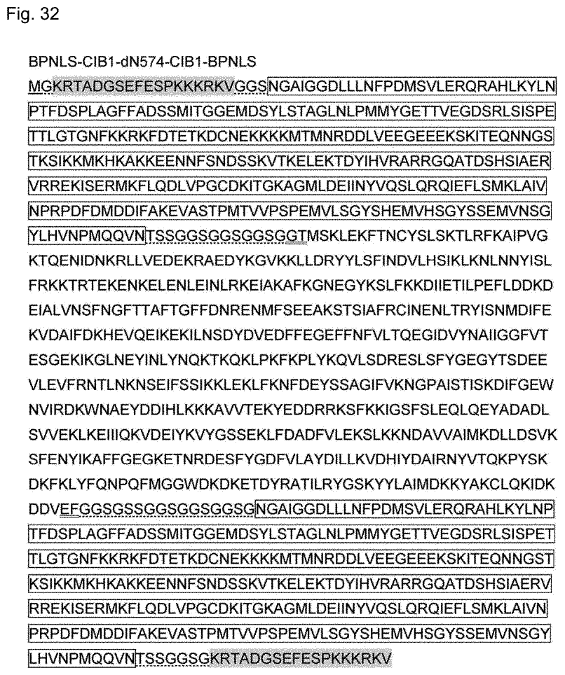

FIG. 33 exhibits the amino acid sequence of BPNLS-CIB1-dC575-NLS containing split-dCpf1-C. In the amino acid sequence, BPNLS is a nuclear localization sequence, NLS in the C-terminal side means Nucleoplasmin NLS, and is a nuclear localization sequence. dC575 is split-dCpf1-C formed by cutting dLbCpf1 at a cutting site of N574/C575. CIB1 is a light switch protein (CRY2-CIB1 system).

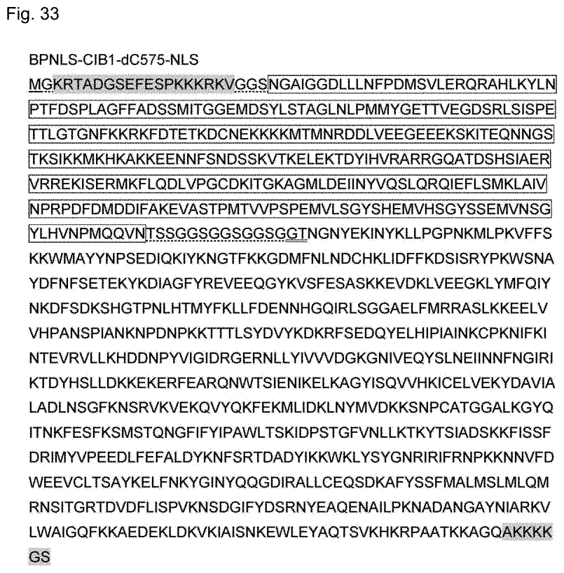

FIG. 34 exhibits the amino acid sequence of NLSx3-CRY2-PHR-p65-HSF1. In the amino acid sequence, NLS means SV40 NLS, and is a nuclear localization sequence, and x3 means three times. CRY2-PHR is a light switch protein (CRY2-CIB1 system). P65 and HSF1 are transcription activation domains.

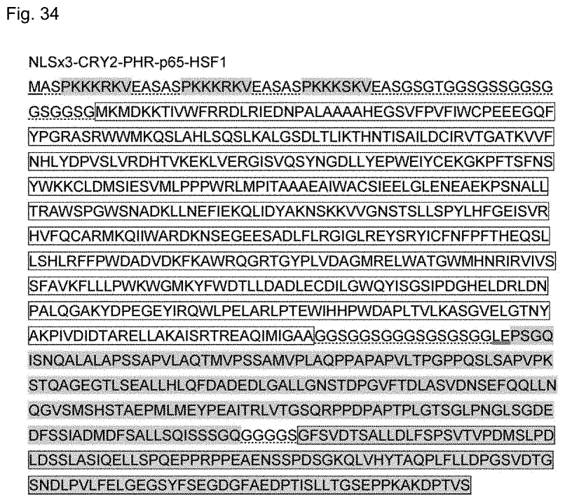

FIG. 35 exhibits the amino acid sequence of BPNLS-p65-HSF1-NLS-dN574-p65-HSF1-BPNLS containing split-Cpf1-N. In the amino acid sequence, BPNLS is a nuclear localization sequence, NLS means SV40 NLS, and is a nuclear localization sequence. N574 is split-dCpf1-N formed by cutting dLbCpf1 at a cutting site of N574/C575. P65 and HSF1 are transcription activation domains.

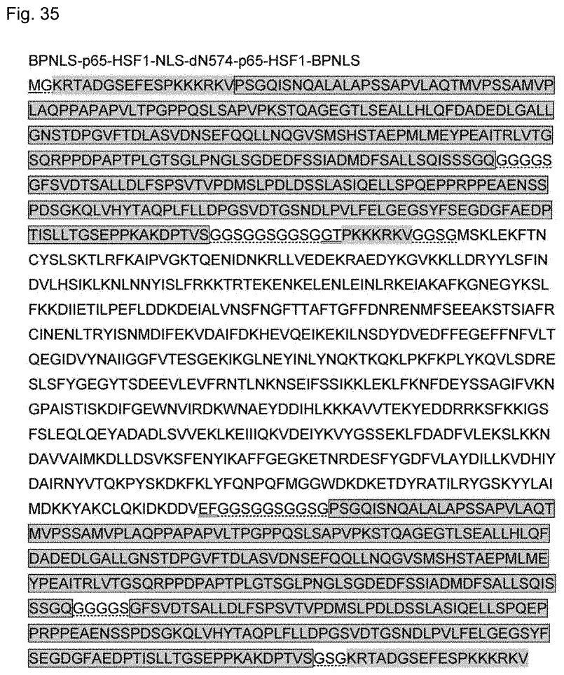

FIG. 36 exhibits the amino acid sequence of BPNLS-p65-HSF1-dC575-p65-HSF1-BPNLS containing split-Cpf1-C. In the amino acid sequence, BPNLS is a nuclear localization sequence. dN574 is split-dCpf1-C formed by cutting dLbCpf1 at a cutting site of N574/C575. P65 and HSF1 are transcription activation domains.

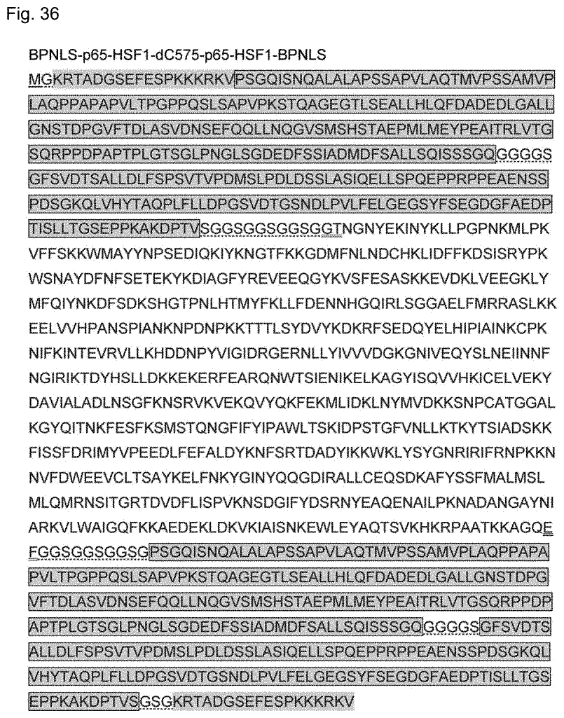

FIG. 37 exhibits comparison on activation efficiency between a divided dCpf1 activator targeting a promoter region and dCas9-SAM. FIGS. 37a to 37e exhibit the comparison results for the promoter regions of ASCL1 (a), IL1R2 (b), AR (c), HBB (d) and IL1RN (e). The cells used were HEK293T cells. In each of FIGS. 37a to 37e, the top panel indicates target sites for the corresponding crRNA and sgRNA, and the specified CRISPR activator (divided dCpf1 activator, dCas9-SAM) and guide RNA (crRNA for the divided dCpf1 activator, sgRNA for dCas9-SAM) were used. The results are represented as relative mRNA levels with respect to a negative control transfected with a vacant vector, and exhibited as average±s.e.m (the number n is 3 for a, c, and d, with three different cell culture samples, and 4 for b and e, with two different separate experimental samples from each of two different cell cultures). Dots indicate individual data points.

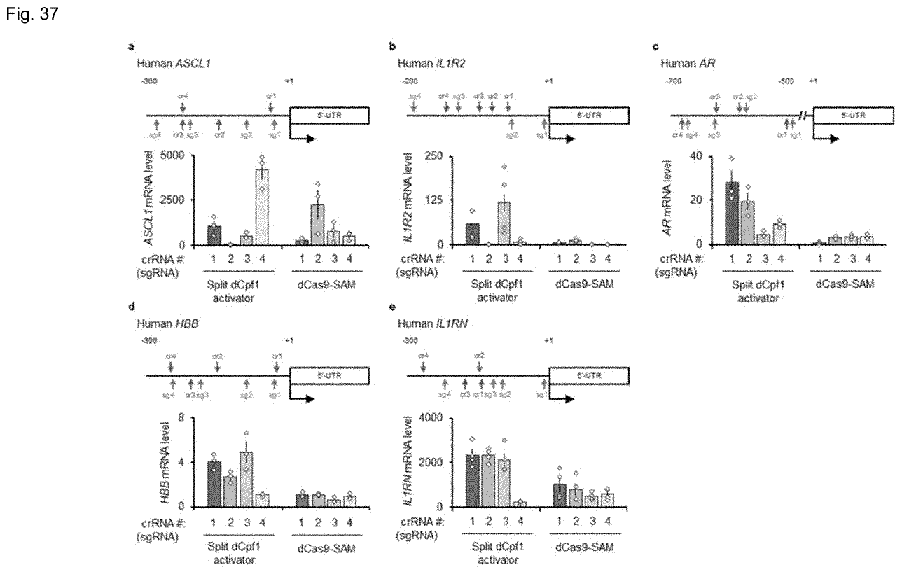

FIG. 38 exhibits in vivo gene activation using a divided dCpf1 activator. FIG. 38a exhibits comparison between the divided dCpf1 activator and a dCpf1-VPR activator in activation of a luciferase reporter in living mice. Plasmids which express a dCpf1 activator (divided dCpf1 activator or dCpf1-VPR activator), a GAL4-UAS luciferase reporter and a crRNA targeting the reporter (or negative crRNA) were delivered from the tail vein to the liver by hydrodynamic injection. Bioluminescence imaging was performed 24 hours after injection. A scale bar indicates 1 cm. FIG. 38b exhibits quantitation of the bioluminescence activities shown in FIG. 38a (the number n is 3). FIG. 38c exhibits endogenous Ascl1 activation using a dCpf1 activator. The data were represented as relative mRNA levels with respect to a negative control without transfection (the number n is 4). The data in FIGS. 38b and 38c were exhibited as average±s.e.m. Dots indicate individual data points. Welch's t test was performed to give P values.

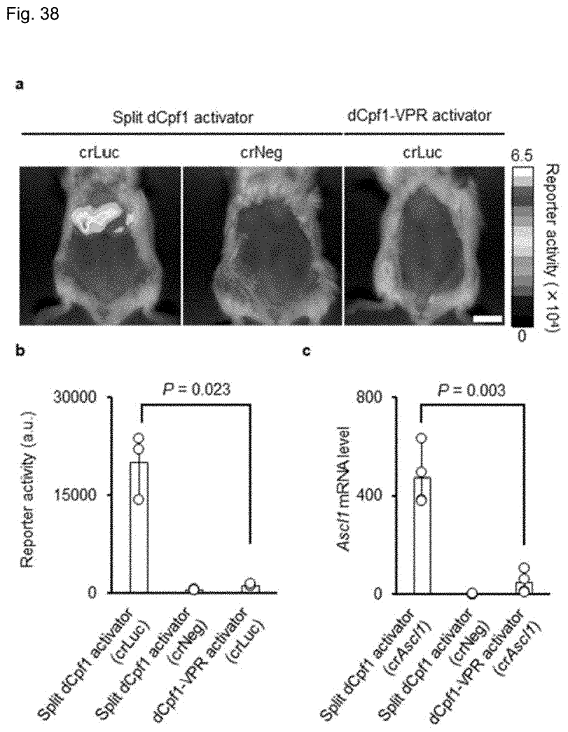

### DESCRIPTION OF EMBODIMENTS

The present invention will be explained more specifically by Description of Embodiments, but the present invention is not limited to the following Description of Embodiments, and can be carried out by various modifications.

(Set of Two Polypeptides of Split Cpf1 Protein)

The set of two polypeptides of a split Cpf1 protein according to the present invention is a set of two polypeptides, in which the two polypeptides are the N-terminal side fragment of a Cpf1 protein and the C-terminal side fragment of the Cpf1 protein.

When a Cpf1 protein is divided into two, two polypeptides are obtained. Of the two polypeptides, the fragment including the N-terminal amino acid of the Cpf1 protein is referred to as the N-terminal side fragment of the Cpf1 protein, and the fragment including the C-terminal amino acid of the Cpf1 protein is referred to as the C-terminal side fragment of the Cpf1 protein.

In the present specification, the Cpf1 protein means Cpf1 and a mutant thereof, and the term is used as meaning including the following (1) to (3):

(1) Cpf1 nuclease containing native Cpf1 and being nuclease-active (simply expressed as “Cpf1 ”, in some cases);
(2) nuclease-inactive mutated Cpf1 (simply expressed as “dead Cpf1 (dCpf1))”, in some cases); and
(3) Cpf1 nickase (nCpf1) as nickase-type mutated Cpf1.

Mutants of naturally occurring Cpf1, dCpf1 and nCpf1, the mutants having mutation, without impairing the original function, in a part being irrelevant to functions, are also included in the Cpf1 protein in the present specification.

dCpf1 and nCpf1 are such mutants of Cpf1 that at least one of the two DNA cutting abilities of Cpf1 has been deactivated.

The set of two polypeptides of a split Cpf1 protein according to the present invention is preferably such that the N-terminal side fragment of the Cpf1 protein and the C-terminal side fragment of the Cpf1 protein are rearranged through spontaneous association.

The set of two polypeptides of a split Cpf1 protein in the present invention is preferably a set of two fused polypeptides of a split Cpf1 protein. In the case of the set of two fused polypeptides, one of the N-terminal side fragment of the Cpf1 protein and the C-terminal side fragment of the Cpf1 protein is bound to each of two polypeptides which form a dimer with dependence on light or in the presence of a drug, and the fused N-terminal side fragment of the Cpf1 protein and the fused C-terminal side fragment of the Cpf1 protein are rearranged through induced association as the two polypeptides which form a dimer with dependence on light or in the presence of a drug form a dimer on being light-induced or drug-induced. Also in the case of the set of two fused polypeptides, the N-terminal side fragment of the Cpf1 protein and the C-terminal side fragment of the Cpf1 protein may be rearranged through spontaneous association.

In the present invention, being rearranged through spontaneous association or induced association means that, spontaneously or on being induced, two polypeptides of a split Cpf1 protein associate to arrange again the properties possessed by the Cpf1 protein before being divided into two.

Examples of the properties of a CPf1 protein for the case where two polypeptides of a split Cpf1 protein are rearranged include nuclease activity, nuclease inactivity, and nickase activity.

(Set of Two Polypeptides of Nuclease-Active Cpf1 Protein)

The set of two polypeptides of a split Cpf1 protein according to the present invention (split-Cpf1) is a set of two polypeptides formed by dividing into the N-terminal side fragment of a Cpf1 protein (split-Cpf1-N) and the C-terminal side fragment thereof (split-Cpf1-C), and the set of two polypeptides is rearranged through induced association or spontaneous association to exhibit nuclease activity.

In the present specification, the nuclease activity means the activity of hydrolyzing and cutting a phosphodiester bond between bases of a double-stranded nucleic acid, which is the original function of Cpf1.

In the present specification, the nuclease-active Cpf1 protein is also expressed as Cpf1.

The set of two polypeptides of a split Cpf1 protein according to the present invention (split-Cpf1) is preferably such a set of two polypeptides that the N-terminal side fragment of a Cpf1 protein (split-Cpf1-N) and the C-terminal side fragment thereof (split-Cpf1-C) spontaneously associate, and the N-terminal side fragment and the C-terminal side fragment of the Cpf1 protein are rearranged through spontaneous association to exhibit nuclease activity.

The set of two polypeptides of a split Cpf1 protein according to the present invention (split-Cpf1) is preferably a set of two fused polypeptides in which one of the N-terminal side fragment of a Cpf1 protein (split-Cpf1-N) and the C-terminal side fragment thereof (split-Cpf1-C) is bound to each of two polypeptides which form a dimer with dependence on light or in the presence of a drug, and the N-terminal side fragment and the C-terminal side fragment of the Cpf1 protein are rearranged to exhibit nuclease activity through induced association with dependence on light or in the presence of a drug.

The “set of two polypeptides of a nuclease-active Cpf1 protein” according to the present invention can precisely cut a target double-stranded nucleic acid sequence by using by combining it with a guide RNA designed based on the target double-stranded nucleic acid sequence. Herein, the guide RNA is also called crRNA, and plays a role in inducing Cpf1 nuclease to a target sequence. The guide RNA used in the present invention may be designed like a guide RNA used in the standard Cpf1 system. For example, it can be designed so as to include a sequence having “TTTV” (V indicates any base of A, C and G) in the 5′-terminal side and being complementary to about 20 to 25 bases from the 3′-terminus of crRNA. By preparing a plurality of guide RNAs, a plurality of target sequences can also be cut at the same time.

Such a method of cutting a double-stranded nucleic acid is also included in the present invention.

Furthermore, when the “set of two polypeptides of a nuclease-active Cpf1 protein” according to the present invention and NHEJ or HDR are combined, desired indel mutation can also be introduced into the target sequence. Multiple gene modification may be performed using a plurality of guide RNAs.

(Set of Two Polypeptides of Nuclease-Inactive Cpf1 Protein)

The set of two polypeptides of a split Cpf1 protein according to the present invention (split-dCpf1) is a set of two polypeptides formed by dividing into the N-terminal side fragment of a Cpf1 protein (split-dCpf1-N) and the C-terminal side fragment thereof (split-dCpf1-C), and the set of two polypeptides is rearranged to become nuclease-inactive through induced association or spontaneous association.

In the present specification, the nuclease-inactive Cpf1 protein is also expressed as dCpf1.

The set of two polypeptides of a split Cpf1 protein according to the present invention (split-dCpf1) is preferably such a set of two polypeptides that the N-terminal side fragment of a Cpf1 protein (split-dCpf1-N) and the C-terminal side fragment thereof (split-dCpf1-C) spontaneously associate, and the N-terminal side fragment and the C-terminal side fragment of the Cpf1 protein are rearranged through spontaneous association.

The set of two polypeptides of a split Cpf1 protein according to the present invention (split-dCpf1) is preferably a set of two fused polypeptides in which one of the N-terminal side fragment and the C-terminal side fragment of a Cpf1 protein is bound to each of two polypeptides which form a dimer with dependence on light or in the presence of a drug, and the N-terminal side fragment and the C-terminal side fragment of the Cpf1 protein are rearranged through induced association with dependence on light or in the presence of a drug. Also in the case of the set of two fused polypeptides, the N-terminal side fragment of the Cpf1 protein and the C-terminal side fragment of the Cpf1 protein may be rearranged through spontaneous association.

The nuclease-inactive Cpf1 protein is obtained, for example, by artificially mutating the amino acid sequence of Cpf1 nuclease. Specifically, it is a mutant formed by mutating an amino acid in the nuclease activity center of Cpf1 nuclease to cause the loss of the nuclease activity, and, for Cpf1 derived from the Lachnospiraceae bacterium ND2006 (LbCpf1) described later, it has a mutation of any one of D832A, E925A and D1180A.

In the present specification, when mutation is contained at a Y position of an amino acid sequence of SEQ ID No.: X, and addition or deletion is generated from a natural sequence in SEQ ID No.: X, which amino acid corresponds to a Y position can be determined by a person skilled in the art, subsequent to sequences before and after etc. Accordingly, in the case of E925A of LbCpf1, for example, an amino acid which is 925th when counted from an N-terminus is not necessarily substituted with A, and it is meant that an amino acid which corresponds to 925th E when counted from an N-terminus in a naturally occurring amino acid sequence is substituted with A.

Thus, LbCpf1 provided with a mutation of any of D832A, E925A and D1180A is regarded as dLbCpf1, and Cpf1 derived from another species can be regarded as dCpf1 if the amino acid of D or E corresponding to D832, E925, or D1180 of LbCpf1 in the Cpf1 derived from another species is substituted with A.

Introduction of any one of D832A, E925A and D1180A allows LbCpf1 to become nuclease-inactive dCpf1, introduction of any one of D908A and E993A allows Cpf1 derived from the Acidaminococcus sp. BV3L6 (AsCpf1) to become nuclease-inactive dCpf1, and introduction of any one of D917A and E1006A allows Cpf1 derived from the Francisella tularensis subsp. novicida U112 (FnCpf1) to become nuclease-inactive dFnCph1.

It is preferable for the set of nuclease-inactive polypeptides (split-dCpf1) that a functional domain be bound to one of the N-terminal side fragment (split-dCpf1-N) and the C-terminal side fragment (split-dCpf1-C) as two polypeptides of the Cpf1 protein.

In the present specification, in the case of the set of nuclease-inactive polypeptides (split-dCpf1), rearranged dCpf1 can exert the function based on the functional domain, especially, activates or suppresses gene expression if a transcription activation domain or a transcriptional repression domain is used as the functional domain.

In the case of the set of nuclease-inactive spontaneously associative polypeptides (split-dCpf1), a functional domain is preferably bound to the N-terminal side fragment (split-dCpf1-N) and/or the C-terminal side fragment (split-dCpf1-C) as two polypeptides of the Cpf1 protein.

A functional domain may be bound to the N-terminus and C-terminus of the N-terminal side fragment (split-dCpf1-N) as one of two polypeptides of the Cpf1 protein and/or the N-terminus and C-terminus of the C-terminal side fragment (split-dCpf1-C) as the other of two polypeptides of the Cpf1 protein. That is, four functional domains may be bound to the set of polypeptides (split-dCpf1).

In the case of the set of nuclease-inactive spontaneously associative polypeptides (split-dCpf1), a functional domain is preferably bound to one of two polypeptides which form a dimer with dependence on light or in the presence of a drug, and the other of two polypeptides which form a dimer with dependence on light or in the presence of a drug is bound to the N-terminus and C-terminus of the N-terminal side fragment (split-dCpf1-N) as one of two polypeptides of the Cpf1 protein and/or the N-terminus and C-terminus of the C-terminal side fragment (split-dCpf1-C) as the other of two polypeptides of the Cpf1 protein.

The set of polypeptides (split-dCpf1) spontaneously associates and two polypeptides which form a dimer with dependence on light or in the presence of a drug form a dimer with dependence on light or in the presence of a drug; accordingly, four functional domains bound to one of the two polypeptides which form a dimer with dependence on light or in the presence of a drug can be present.

A functional domain may be directly bound to the N-terminus and C-terminus of the N-terminal side fragment (split-dCpf1-N) as one of two polypeptides of the Cpf1 protein and/or the N-terminus and C-terminus of the C-terminal side fragment (split-dCpf1-C) as the other of two polypeptides of the Cpf1 protein, without through two polypeptides which form a dimer with dependence on light or in the presence of a drug. In this case, in the set of polypeptides (split-dCpf1), a functional domain is directly bound to the N-terminus and C-terminus of the N-terminal side fragment (split-dCpf1-N) as one of two polypeptides of the Cpf1 protein and/or the N-terminus and C-terminus of the C-terminal side fragment (split-dCpf1-C) as the other of two polypeptides of the Cpf1 protein, without through two polypeptides which form a dimer with dependence on light or in the presence of a drug, and bound to one of two polypeptides which form a dimer with dependence on light or in the presence of a drug, and the other of two polypeptides which form a dimer with dependence on light or in the presence of a drug is bound to the N-terminus and C-terminus of the N-terminal side fragment (split-dCpf1-N) as one of two polypeptides of the Cpf1 protein and/or the N-terminus and C-terminus of the C-terminal side fragment (split-dCpf1-C) as the other of two polypeptides of the Cpf1 protein.

In the case of the set of nuclease-inactive inducibly associative polypeptides (split-dCpf1), a functional domain is preferably bound to the N-terminal side fragment (split-dCpf1-N) and/or the C-terminal side fragment (split-dCpf1-C) as two polypeptides of the Cpf1 protein.

A functional domain may be bound to the N-terminus and C-terminus of the N-terminal side fragment (split-dCpf1-N) as one of two polypeptides of the Cpf1 protein and/or the N-terminus and C-terminus of the C-terminal side fragment (split-dCpf1-C) as the other of two polypeptides of the Cpf1 protein. That is, two functional domains may be bound to the set of polypeptides (split-dCpf1).

The set of polypeptides (split-dCpf1) exerts the function based on a functional domain in a target double-stranded nucleic acid sequence by using by combining it with a guide RNA designed based on the target double-stranded nucleic acid sequence.

Such a method of exerting the function based on a functional domain in a double-stranded nucleic acid is also included in the present invention.

In the case of the set of nuclease-inactive inducibly associative polypeptides (split-dCpf1), a functional domain may be bound to two polypeptides which form a dimer with dependence on light or in the presence of a drug and are bound to the two polypeptides of the Cpf1 protein, and may be additionally bound to the N-terminus and C-terminus of the N-terminal side fragment (split-dCpf1-N) and/or the N-terminus and C-terminus of the C-terminal side fragment (split-dCpf1-C) as two polypeptides of the Cpf1 protein.

If rearrangement is achieved through spontaneous association, a set of fused polypeptides to which a plurality of functional domains is bound can be employed.

Examples of functional domains in the present invention include transcription activation domains, transcriptional repression domains, recombinases, deaminases, epigenetic modifiers, and nucleases.

The transcription activation domain, a domain also called a transactivation domain or a transactivator, is a transcription activation domain for a target gene. Examples of the transcription activation domain include VP16, VP64, p65 and HSF1.

Examples of the transcriptional repression domain include KRAB and SID4X.

Examples of the recombinase include serine recombinase (e.g., Hin, Gin or Tn3 recombinase) and tyrosine recombinase (e.g., Cre recombinase).

Examples of the deaminase include cytidine deaminase (e.g., APOBEC1, AID or ACF1/ASE deaminase) and adenosine deaminase (e.g., ADAT family deaminase).

Examples of the epigenetic modifier include histone demethylase, histone methyltransferase, hydroxylase, histone deacetylase, and histone acetyltransferase.

Examples of the nuclease include exonuclease (e.g., TREX2, TREX2, Exol, lambda exonuclease) and endonuclease (e.g., FokI).

The set of nuclease-inactive polypeptides can be designed in the same manner as for the N-terminal side fragment and the C-terminal side fragment of the Cpf1 protein to be used for the set of nuclease-active polypeptides.

Binding of a functional domain to the N-terminal side fragment and/or the C-terminal side fragment of the Cpf1 protein, as well as to two polypeptides which form a dimer with dependence on light or in the presence of a drug are achieved through a linker or without through a linker.

If binding is achieved through a linker, a flexible linker comprising one or more glycine and serine as constituent amino acids can be used therefor.

The set of polypeptides according to the present invention, if a functional domain therein is a transcription activation domain or a transcriptional repression domain, activates or suppresses expression of a target gene.

In the present specification, “expression of a gene” is used as the concept including both of transcription by which an RNA is synthesized employing a DNA as a template, and translation by which a polypeptide is synthesized based on an RNA sequence.

The set of two polypeptides which is a set of nuclease-inactive inducibly associative polypeptides (split-dCpf1) and activates or suppresses expression of a target gene can activate or suppress expression of the target gene by combining it with a guide RNA having a sequence complementary to a part of a sequence of the target gene. In this case, the guide RNA can have, for example, a sequence complementary to a part (e.g. about 20 nucleotides) of a promoter sequence or an exon sequence of a sense strand or an antisense strand of the target gene, thereby, initiation of transcription or elongation of a mRNA is inhibited.

Such a method of activating or suppressing gene expression is also included in the present invention.

In the present invention, as the transcription activation domain, a set of two polypeptides activating gene expression of the target gene, containing a polypeptide in which VP64 is bound to the C-terminal side fragment of the Cpf1 protein is preferable, and it is suitable that as an aptamer-binding protein, MS2 is used, and as the transcription activation domain binding to the aptamer-binding protein, p65 and HSF1 are used.

As a factor corresponding to VP64, MS2, p65 and HSF1, the known transcription activation domain and aptamer-binding protein can also be used, and for example, a transcription activation domain and an aptamer-binding protein such as those disclosed in Nature (2015) 517, 583-588 and Nature protocols (2012) 7 (10), 1797-1807 can be used.

(Set of Two Polypeptides of Nickase-Active Cpf1 Protein)

The set of two polypeptides of a split Cpf1 protein according to the present invention (split-nCpf1) is a set of two polypeptides formed by dividing into the N-terminal side fragment of a Cpf1 protein (split-dCpf1-N) and the C-terminal side fragment thereof (split-dCpf1-C), and the set of two polypeptides is rearranged to exhibit nuclease activity through induced association or spontaneous association.

In the present specification, the nickase activity means the activity of forming a nick in a single strand among a double-stranded nucleic acid.

In the present specification, the nickase-active Cpf1 protein is also expressed as nCpf1.

The set of nickase-active polypeptides can be designed in the same manner as for the N-terminal side fragment and the C-terminal side fragment of the Cpf1 protein to be used for the set of nuclease-active polypeptides.

Like the set of nuclease-inactive polypeptides, the set of nickase-active polypeptides may be a set of polypeptides having a functional domain such as a transcription activation domain and deaminase.

The set of two polypeptides exhibiting the nickase activity can cut a target double-stranded nucleic acid by combining it with a pair of guide RNAs targeting each strand of the target double-stranded nucleic acid. In this case, since the target double-stranded nucleic acid is cut at a region sandwiched by a pair of guide RNAs, sequence specificity can be enhanced more than the case where a single guide RNA is used.

Each guide RNA can be designed like the set of nuclease-active polypeptides. Alternatively, by preparing a plurality of pairs of guide RNAs, a plurality of target sequences can also be cut at the same time.

Such a method of cutting a double-stranded nucleic acid is also included in the present invention.

Additionally, when the “set of two polypeptides of nickase-active Cpf1 protein ” according to the present invention is combined with NHEJ or HDR, desired indel mutation can also be introduced into the target sequence. Multiple gene modification may be performed using a plurality of guide RNAs.

The nickase-active Cpf1 protein is obtained, for example, by artificially mutating the amino acid sequence of Cpf1 nuclease. Specifically, it is a mutant formed by mutating an amino acid in the nuclease activity center of Cpf1 nuclease to cause the loss of the nuclease activity, and, for example, it contains a mutation of R1138A for LbCpf1, and a mutation of R1226A for AsCpf1.

Accordingly, in the case of R1138A of LbCpf1, for example, an amino acid which is 1138th when counted from an N-terminus is not necessarily substituted with A, and it is meant that an amino acid which corresponds to 1138th R when counted from an N-terminus in a naturally occurring amino acid sequence is substituted with A.

Thus, LbCpf1 provided with a mutation of R1138A is regarded as nLbCpf1, and Cpf1 derived from another species can be regarded as nCpf1 if the amino acid corresponding to R1138 of LbCpf1 in the Cpf1 derived from another species is substituted with A.

In the present invention, the N-terminal side fragment and the C-terminal side fragment of the Cpf1 protein may be each a fragment comprising a partial sequence of the Cpf1 protein or a sequence containing mutation in such a partial sequence.

Although the following description uses SEQ ID No.: 2, which is the full length amino acid sequence of LbCpf1, as an example, the amino acids corresponding to the amino acid sequence of LbCpf1 may be selected even for Cpf1 derived from another species.

An N-terminal amino acid of the N-terminal side fragment is an amino acid which is more on a side of N-terminal than an N-terminal amino acid of the C-terminal side fragment, in a sequence of SEQ ID No.: 2. A C-terminal amino acid of the N-terminal side fragment may be an amino acid which is more N-terminal, or more C-terminal than an N-terminal amino acid of the C-terminal side fragment, in the sequence of SEQ ID No.: 2.

The N-terminal side fragment and the C-terminal side fragment may be designed that a region in which the N-terminal side fragment or the C-terminal side fragment and an amino acid sequence of SEQ ID No.: 2 are overlapped becomes 70% or more, 80% or more, 90% or more, 95% or more, 98% or more, 100%, or 100% or more of an amino acid sequence of SEQ ID No.: 2. Herein, the “region in which the N-terminal side fragment or the C-terminal side fragment and an amino acid sequence of SEQ ID No.: 2 are overlapped” means, for example, 990 amino acids of from a 11-positional amino acid to a 1000-positional amino acid, when the N-terminal side fragment is composed of from a 11-positional amino acid to a 400-positional amino acid of SEQ ID No.: 2, and the C-terminal side fragment is composed of from a 401-positional amino acid to a 1000-positional amino acid. Accordingly, the relevant region is about 78% of an amino acid sequence (1273 amino acids) of SEQ ID No.: 2. Additionally, for example, the “region in which the N-terminal side fragment or the C-terminal side fragment and an amino acid sequence of SEQ ID No.: 2 are overlapped” is composed of 1180 amino acids which is a total of 590 amino acids of from 11-positional to 600-positional amino acids, and 590 amino acids of from position 611 to position 1200, and is about 93% of an amino acid sequence of SEQ ID No.: 2, when the N-terminal side fragment is composed of from a 11-positional amino acid to a 600-positional amino acid of SEQ ID No.: 2, and the C-terminal side fragment is composed of from a 611-positional amino acid to a 1200-positional amino acid.

The N-terminal side fragment or the C-terminal side fragment obtained by designing so that a region, in which the N-terminal side fragment or the C-terminal side fragment of Cpf1, and an amino acid sequence of SEQ ID No.: 2 are overlapped, becomes 70% or more, 80% or more, 90% or more, 95% or more, 98% or more, 100%, or 100% or more of an amino acid sequence of SEQ ID No.: 2 can become an N-terminal side fragment or a C-terminal side fragment in Cpf1 or a Cpf1 protein derived from other species other than derived from Lachnospiraceae bacterium ND2006. An N-terminal side fragment or a C-terminal side fragment in Cpf1 or a Cpf1 protein derived from other species other than derived from the Lachnospiraceae bacterium ND2006 may be split Cpf1 or a split Cpf1 protein formed, with reference to a cutting site giving an N-terminal side fragment and a C-terminal side fragment for LbCpf1, by dividing at the corresponding site.

In the present specification, the same applies in the case of a fragment comprising an amino acid sequence containing addition, substitution or deletion of one to several amino acids, or a fragment comprising an amino acid sequence having 80% or more sequence identity with an amino acid sequence of a fragment.

In the present invention, Table 1 shows examples of Cpf1 which can be used in place of LbCpf1 derived from the Lachnospiraceae bacterium ND2006.

TABLE 1

| | |
|---|---|
| 1. | Francisella tularensis subsp. Novicida U112 Cpf1 (FnCpf1) |
| 2. | Lachnospiraceae bacterium MC2017 Cpf1 (Lb3Cpf1) |
| 3. | Butyrivibrio proteoclasticus Cpf1 (BpCpf1) |
| 4. | Peregrinibacteria bacterium GW2011_GWA_33_10 Cpf1 |
| | (PeCpf1) |
| 5. | Parcubacteria bacterium GWC2011_GWC2_44_17 Cpf1 (PbCpf1) |
| 6. | Smithella sp. SC_K08D17 Cpf1 (SsCpf1) |
| 7. | Acidaminococcus sp. BV3L6 Cpf1 (AsCpf1) |
| 8. | Lachnospiraceae bacterium MA2020 Cpf1 (Lb2Cpf1) |
| 9. | Candidatus Methanoplasma termitum Cpf1 (CMtCpf1) |
| 10. | Eubacterium eligens Cpf1 (EeCpf1) |
| 11. | Moraxella bovoculi 237 Cpf1 (MbCpf1) |
| 12. | Leptospira inadai Cpf1 (LiCpf1) |
| 13. | Lachnospiraceae bacterium ND2006 Cpf1 (LbCpf1) |
| 14. | Porphyromonas crevioricanis Cpf1 (PeCpf1) |
| 15. | Prevotella disiens Cpf1 (PdCpf1) |
| 16. | Porphyromonas macacae Cpf1 (PmCpf1) |

The N-terminal side fragment and the C-terminal side fragment may be designed as a fragment comprising 100 or more amino acids, 200 or more amino acids, 300 or more amino acids, 400 or more amino acids, 500 or more amino acids, 600 or more amino acids, or 700 or more amino acids of an amino acid sequence of SEQ ID No.: 2, respectively.

It is preferable that the N-terminal side fragment and the C-terminal fragment are cut at a domain other than nuclease domains (RuvC or UK) involved in DNA cutting, in an amino acid sequence of SEQ ID No.: 2, and it is preferable to cut at a region jointing an α-helix or a β-sheet (e.g., a loop region) and oriented to the outer side of the Cpf1 molecule.

The N-terminal side fragment and the C-terminal fragment may be, for example, fragments obtained by cutting an amino acid sequence of SEQ ID No.: 2 at any position of position 69 to position 73, position 83 to position 89, position 131 to position 138, position 244 to position 252, position 265 to position 296, position 309 to position 312, position 371 to position 387, position 404 to position 409, position 437 to position 445, position 549 to position 552, position 567 to position 577, position 606 to position 609, position 619 to position 628, position 727 to position 736, position 802 to position 811, position 1037 to position 1042, position 1140 to position 1148, position 1155 to position 1161, and position 1163 to position 1178.

In the case of the inducibly associative type, the N-terminal side fragment and the C-terminal fragment may be fragments obtained by cutting an amino acid sequence of SEQ ID No.: 2 preferably at any position of position 69 to position 73, position 83 to position 89, position 131 to position 138, position 244 to position 252, position 265 to position 296, position 309 to position 312, position 549 to position 552, position 619 to position 628, position 727 to position 736, position 802 to position 811, position 1037 to position 1042, position 1140 to position 1148, position 1155 to position 1161, and position 1163 to position 1178, more preferably at any position of position 309 to position 312, position 549 to position 552, position 727 to position 736, position 1037 to position 1042, and position 1163 to position 1178, furthermore preferably at a position of position 309 to position 312 or position 727 to position 736.

In the case of the spontaneously associative type, the N-terminal side fragment and the C-terminal fragment may be fragments obtained by cutting an amino acid sequence of SEQ ID No.: 2 preferably at any position of position 83 to position 89, position 244 to position 252, position 371 to position 387, position 404 to position 409, position 437 to position 445, position 567 to position 577, and position 606 to position 609, more preferably at any position of position 371 to position 387, position 404 to position 409, position 437 to position 445, position 567 to position 577, and position 606 to position 609, furthermore preferably at a position of position 567 to position 577.

The N-terminal side fragment and the C-terminal fragment may be a fragment comprising an amino acid sequence containing addition, substitution, or deletion of one to several amino acids, in an amino acid sequence of the thus obtained fragment, or a fragment comprising an amino acid sequence having 80% or more sequence identity with an amino acid sequence of the thus obtained fragment.

The N-terminal side fragment and the C-terminal side fragment of the Cpf1 protein may be a fragment comprising a sequence of 50 to 1223 amino acids containing an N-terminus in an amino acid sequence of SEQ ID No.: 2, and a fragment comprising a sequence of 50 to 1223 amino acids containing a C-terminus in an amino acid sequence of SEQ ID No.: 2, respectively.

The N-terminal side fragment and the C-terminal side fragment may be a fragment comprising an amino acid sequence containing addition, substitution, or deletion of one to several amino acids, in an amino acid sequence of such a fragment, or a fragment comprising an amino acid sequence having 80% or more sequence identity with an amino acid sequence of such a fragment.

The N-terminal side fragment and the C-terminal side fragment of the Cpf1 protein may be any of the following combinations:

a combination of an N-terminal fragment comprising amino acids at position 1 to position 70 in an amino acid sequence of SEQ ID No.: 2, and a C-terminal fragment comprising amino acids at position 71 to position 1273;

a combination of an N-terminal fragment comprising amino acids at position 1 to position 86 in an amino acid sequence of SEQ ID No.: 2, and a C-terminal fragment comprising amino acids at position 87 to position 1273;

a combination of an N-terminal fragment comprising amino acids at position 1 to position 134 in an amino acid sequence of SEQ ID No.: 2, and a C-terminal fragment comprising amino acids at position 135 to position 1273;

a combination of an N-terminal fragment comprising amino acids at position 1 to position 248 in an amino acid sequence of SEQ ID No.: 2, and a C-terminal fragment comprising amino acids at position 249 to position 1273;

a combination of an N-terminal fragment comprising amino acids at position 1 to position 266 in an amino acid sequence of SEQ ID No.: 2, and a C-terminal fragment comprising amino acids at position 267 to position 1273;

a combination of an N-terminal fragment comprising amino acids at position 1 to position 310 in an amino acid sequence of SEQ ID No.: 2, and a C-terminal fragment comprising amino acids at position 311 to position 1273;

a combination of an N-terminal fragment comprising amino acids at position 1 to position 373 in an amino acid sequence of SEQ ID No.: 2, and a C-terminal fragment comprising amino acids at position 374 to position 1273;

a combination of an N-terminal fragment comprising amino acids at position 1 to position 406 in an amino acid sequence of SEQ ID No.: 2, and a C-terminal fragment comprising amino acids at position 407 to position 1273;

a combination of an N-terminal fragment comprising amino acids at position 1 to position 443 in an amino acid sequence of SEQ ID No.: 2, and a C-terminal fragment comprising amino acids at position 444 to position 1273;

a combination of an N-terminal fragment comprising amino acids at position 1 to position 550 in an amino acid sequence of SEQ ID No.: 2, and a C-terminal fragment comprising amino acids at position 551 to position 1273;

a combination of an N-terminal fragment comprising amino acids at position 1 to position 574 in an amino acid sequence of SEQ ID No.: 2, and a C-terminal fragment comprising amino acids at position 575 to position 1273;

a combination of an N-terminal fragment comprising amino acids at position 1 to position 607 in an amino acid sequence of SEQ ID No.: 2, and a C-terminal fragment comprising amino acids at position 608 to position 1273;

a combination of an N-terminal fragment comprising amino acids at position 1 to position 624 in an amino acid sequence of SEQ ID No.: 2, and a C-terminal fragment comprising amino acids at position 625 to position 1273;

a combination of an N-terminal fragment comprising amino acids at position 1 to position 730 in an amino acid sequence of SEQ ID No.: 2, and a C-terminal fragment comprising amino acids at position 731 to position 1273;

a combination of an N-terminal fragment comprising amino acids at position 1 to position 808 in an amino acid sequence of SEQ ID No.: 2, and a C-terminal fragment comprising amino acids at position 809 to position 1273;

a combination of an N-terminal fragment comprising amino acids at position 1 to position 1039 in an amino acid sequence of SEQ ID No.: 2, and a C-terminal fragment comprising amino acids at position 1040 to position 1273;

a combination of an N-terminal fragment comprising amino acids at position 1 to position 1143 in an amino acid sequence of SEQ ID No.: 2, and a C-terminal fragment comprising amino acids at position 1144 to position 1273;

a combination of an N-terminal fragment comprising amino acids at position 1 to position 1157 in an amino acid sequence of SEQ ID No.: 2, and a C-terminal fragment comprising amino acids at position 1158 to position 1273;

a combination of an N-terminal fragment comprising amino acids at position 1 to position 1170 in an amino acid sequence of SEQ ID No.: 2, and a C-terminal fragment comprising amino acids at position 1171 to position 1273; and

a combination containing addition, substitution, or deletion of one to several amino acids in a sequence of at least one fragment, in any of the aforementioned combinations; as well as

a combination in which a sequence of at least one fragment is a fragment having 80% or more sequence identity with the above sequence, in any of the aforementioned combinations.

As a specific example of the N-terminal side fragment and the C-terminal fragment for which it is preferable to cut at a domain other than nuclease domains (RuvC or UK) involved in DNA cutting described above, and it is preferable to cut at a region jointing an α-helix or a β-sheet (e.g., a loop region) and oriented to the outer side of the Cpf1 molecule, any of the above combinations may be selected. In the case of the inducibly associative type or the spontaneously associative type, likewise, any of the above combinations may be selected.

In the present specification, an amino acid, an “amino acid” is used in its broadest sense, and includes, in addition to a natural amino acid, a derivative thereof or an artificial amino acid. In the present specification, examples of an amino acid include a natural proteinaceous L-amino acid; a non-natural amino acid; a chemically synthesized compound having the properties known in the art, which are the characteristics of an amino acid. Examples of the non-natural amino acid are not limited to, but include an α, α-disubstituted amino acid (α-methylalanine etc.), an N-alkyl-α-amino acid, a D-amino acid, a β-amino acid, and a α-hydroxyacid, in which a structure of a main chain is different from a natural type, an amino acid in which a structure of a side chain is different from a natural type (norleucine, homohistidine etc.), an amino acid in which a side chain has extra methylene (“homo”amino acid, homophenylalanine, homohistidine etc.), and an amino acid in which a carboxylic acid functional group amino acid in a side chain is substituted with a sulfonic acid group (cysteic acid etc.).

In the present specification, an amino acid is represented by conventional one letter code or three letter code, in some cases. An amino acid represented by one letter code or three letter code includes a mutant and a derivative of each of them, in some cases.

In the present specification, when a certain amino acid sequence contains addition, substitution, or deletion of one to several amino acids, this means that 1, 2, 3, 4, 5, 6, 7, 8 or 9 amino acids are added (inserted), substituted, or deleted at a terminus or a non-terminus of the sequence. The number of amino acids to be added, substituted, or deleted is not particularly limited, as far as the resultant polypeptide exerts the effect in the present invention. Additionally, a site to be added, substituted, or deleted may be at one place or two or more places.

In the present specification, when sequence identity with a certain amino acid sequence is 80% or more, the sequence identity may be 85% or more, 90% or more, 95% or more, 98% or more, or 99% or more. The sequence identity can be obtained by a person skilled in the art according to the known method.

(Set of Two Polypeptides Forming a Dimer with Dependence on Light)

In the present specification, the “set of two polypeptides forming a dimer with dependence on light” (hereinafter, referred to as “light switch”) refers to a pair of natural proteins forming a homodimer or a heterodimer by irradiation of light, or one obtained by artificially modifying this. Non-limiting examples of the light switch include the following:

[Pair Forming a Heterodimer]

PhyB and PIF (Levskaya, A., et al., Nature, 461, 997-1001 (2009).)

FKF1 and GI (Yazawa, M. et al., Nat. Biotechnol. 27, 941-5 (2009).)

CRY2 and CIB1 (Kennedy, M. J., et al., Nat. methods 7, 12-16 (2010).)

UVR8-COP1 (Crefcoeur, R P. et al., Nat. Commun. 4:1779 doi: 10. 1038/ncomms2800 (2013).)

VVD-WC1 (Malzahn, E. et al., Cell, 142, 762-772 (2010).)

PhyB-CRY1 (Hughes, R. M. et al., J. Biol. Chem. 287, 22165-22172 (2012).)

RpBphP1-RpPpsR2 (Bellini, D. et al., Structure, 20, 1436-1446 (2012).)

[Pair Forming a Homodimer]

UVR8 (Chen, D. A. et al., J. Cell Biol. 201, 631-640 (2013).)

EL222 (Motta-Mena, L. B. et al., Nat. Chem. Biol., 10, 196-202 (2014).)

bPac (Stierl, M. et al., Beggiatoa, J. Biol. Chem., 286, 1181-1188 (2001).)

RsLOV (Conrad, K. S. et al., Biochemistry, 52, 378-391 (2013).)

PYP (Fan, H. Y. et al., Biochemistry, 50, 1226-1237 (2011).)

H-NOXA (Zoltowski, B. D. et al., Biochemistry, 47, 7012-7019 (2008).)

YtvA (Zoltowski, B. D. et al., Biochemistry, 47, 7012-7019 (2008).)

NifL (Zoltowski, B. D. et al., Biochemistry, 47, 7012-7019 (2008).)

FixL (Zoltowski, B. D. et al., Biochemistry, 47, 7012-7019 (2008).)

RpBphP1 (Bellini, D. et al., Structure, 20, 1436-1446 (2012).)

CRY2 (Multimer formation) (Zoltowski, B. D. et al., Biochemistry, 47, 7012-7019 (2008).)

In the light switch, the amino acid number of each of the pair may be about 200 or less, about 180 or less, or about 160 or less.

As the light switch, a Magnet which was developed by the present inventors based on the Vivid protein may be used. The Magnet is a set of two different polypeptides which are independently selected from a polypeptide comprising an amino acid sequence of SEQ ID No.: 1, and a mutant polypeptide thereof. Particularly, there is mentioned one having a sequence where one of polypeptides of the set has a sequence in which Ile at a position 52 and Met at a position 55 are substituted with an amino acid having a positive charge on a side chain, in an amino acid sequence of SEQ ID No.: 1 or a sequence having 80% or more, 85% or more, 90% or more, 95% or more, 98% or more, or 99% or more sequence identity with this, and the other polypeptide has a sequence in which Ile at a position 52 and Met at a position 55 are substituted with an amino acid having a negative charge on a side chain, in an amino acid sequence of SEQ ID No.: 1 or a sequence having 80% or more, 85% or more, 90% or more, 95% or more, 98% or more, or 99% more sequence identity with this.

Herein, the amino acid having a positive charge on a side chain may be a natural amino acid or a non-natural amino acid, and examples of the natural amino acid include lysine, arginine, and histidine. The amino acid having a negative charge on a side chain may be also a natural amino acid or a non-natural amino acid, and examples of the natural amino acid include aspartic acid and glutamic acid.

Specific examples of the Magnet include the following:

pMag and nMag

pMag and nMagHigh1

pMagHigh1 and nMag

pMagHigh1 and nMagHigh1.

Herein, pMag refers to a polypeptide having mutations of I52R and M55R, in an amino acid sequence of SEQ ID No.: 1 or a sequence having 80% or more, 85% or more, 90% or more, 95% or more, 98% or more, or 99% or more sequence identity with this, and pMagHigh1 refers to a polypeptide further containing mutations of M135I and M165I, in the amino acid sequence of pMag.

Additionally, nMag refers to a polypeptide having mutations of I52D and M55G, in an amino acid sequence of SEQ ID NO.: 1 or a sequence having 80% or more, 85% or more, 90% or more, 95% or more, 98% or more, or 99% or more sequence identity with this, and nMagHigh1 refers to a polypeptide further containing mutations of M135I and M165I, in the amino acid sequence of nMag.

The Magnet forms a heterodimer by irradiating blue light, and the heterodimer is rapidly dissociated by stopping light irradiation.

Each polypeptide of the light switch, and the N-terminal side fragment and the C-terminal side fragment of the Cpf1 protein can be bound by the known method. Examples thereof include a method of appropriately ligating nucleic acids encoding each of them, and expressing the ligated nucleic acids as a fused polypeptide. In this case, a polypeptide being a linker may intervene between any polypeptide of the light switch, and the N-terminal side fragment or the C-terminal side fragment.

For the linker, for example, a flexible linker comprising one or more glycine and serine as constituent amino acids can be used.

(Set of Two Polypeptides Forming a Dimer in the Presence of a Drug)

The “set of two polypeptides forming a dimer in the presence of a drug” used in the present invention may be the known one. Examples thereof are not limited to, but include a set of FKBP (FK506-binding protein) and FRB (FKBP12-rapamycin associated protein 1 fragment) forming a heterodimer in the presence of rapamycin, a system using gibberellin (compound) and its binding protein (GAI/GID1) (Nat. Chem. Biol. 8, 465-470 (2012) doi: 10.1038/nchembio.922), a system using fusicoccin (compound) and its binding protein (CT52M1/T14-3-3c□C-M2) (PNAS 110, E377-386 (2013) doi: 10.1073/pnas. 1212990110), a system using abscisic acid (compound) and its binding protein (PYL/ADI) (Science Signaling 4(164), rs2 (2011) DOI: 10. 1126/scisignal.2001449), and a system using rCD1/FK506 (compound) and its binding protein (FKBP/SNAP) (Angew. Chem. Int. Ed. 53, 1-5 (2014) DOI: 10.1002/anie.201402294).

Each of the polypeptides forming a dimer in the presence of a drug, and the N-terminal side fragment and the C-terminal side fragment of the Cpf1 protein can be bound as in the case of the light switch.

In binding between polypeptides forming a dimer in the presence of a drug or polypeptides of a light switch protein and the N-terminal side fragment and the C-terminal fragment of the Cpf1 protein, each of the polypeptides can be arbitrarily selected from the corresponding polypeptides shown in the present specification, and the N-terminal side fragment and the C-terminal fragment of the Cpf1 protein can be arbitrarily selected from the fragments and combinations shown in the present specification. That is, in the present specification, any of the polypeptides shown as examples and any of the fragments shown as examples can be bound together, and they can be selected from preferable materials for both, or from preferable materials for one and from more preferable materials for the other. Needless to say, a preferable material and a preferable material may be combined, a preferable material and a more preferable material may be combined, and an exemplified material, a preferable material, a more preferable material, and a furthermore preferable material may be combined.

(Nucleic Acid)

The present invention also provides a nucleic acid encoding a polypeptide constituting the set of two polypeptides in accordance with first to fourth aspects.

A term “nucleic acid” in the present specification includes a DNA, an RNA, a chimera of DNA/RNA, and an artificial nucleic acid such as a locked nucleic acid (LNA) and a peptide nucleic acid (PHA), unless particularly described.

Examples of such a nucleic acid include a nucleic acid encoding a fused polypeptide of one polypeptide of the light switch, and the N-terminal side fragment of the Cpf1 protein, and a nucleic acid encoding a fused polypeptide of the other polypeptide of the light switch, and the C-terminal side fragment of the Cpf1 protein. The nucleic acid may be a nucleic acid encoding a linker polypeptide between any one polypeptide of the light switch, and a fused polypeptide of the N-terminal side fragment or the C-terminal side fragment of the Cpf1 protein.

Additionally, other examples of the nucleic acid according to the present invention include a nucleic acid encoding a fused polypeptide of one of polypeptides forming a dimer in the presence of a drug and the N-terminal side fragment of the Cpf1 protein, and a nucleic acid encoding a fused polypeptide of the other of polypeptides forming a dimer in the presence of a drug and the C-terminal side fragment of the Cpf1 protein. The nucleic acid may be a nucleic acid encoding a linker polypeptide between any one of the set of polypeptides forming a dimer in the presence of a drug, and a fused polypeptide of the N-terminal side fragment or the C-terminal side fragment of the Cpf1 protein.

The nucleic acid according to the present invention can be synthesized according to the known method by a person skilled in the art.

The present invention also includes an expression vector including the nucleic acid according to the present invention. In the expression vector according to the present invention, any one of nucleic acids encoding each of the set of two polypeptides according to the present invention may be inserted, or both of the nucleic acids may be inserted into one vector. Additionally, such a vector may contain a nucleic acid encoding a guide RNA.

The nucleic acid according to the present invention as it is, or after digestion with a restriction enzyme, or addition of a linker, can be inserted downstream of a promoter of the expression vector. Examples of the vector are not limited to, but include an Escherichia coli-derived plasmid (pBR322, pBR325, pUC12, pUC13, pUC18, pUC19, pUC118, pBluescript II etc.), a Bacillus subtilis-derived plasmid (pUB110, pTP5, pC1912, pTP4, pE194, pC194 etc.), a yeast-derived plasmid (pSH19, pSH15, YEp, YRp, YIp, YAC etc.), a bacteriophage (□ phage, M13 phage etc.), a virus (retrovirus, vaccinia virus, adenovirus, adeno-associated virus (AAV), cauliflower mosaic virus, tobacco mosaic virus, baculovirus etc.), a cosmid and the like.

The promoter can be appropriately selected depending on a kind of a host. When the host is an animal cell, for example, a SV40 (simian virus 40)-derived promoter, and a CMV (cytomegalovirus)-derived promoter can be used. When the host is Escherichia coli, a trp promoter, a T7 promoter, a lac promoter and the like can be used.

In the expression vector, a DNA replication origin (ori), a selection marker (antibiotic resistance, auxotrophy etc.), an enhancer, a splicing signal, a polyA addition signal, a nucleic acid encoding a tag (FLAG, HA, GST, GFP etc.) and the like may be integrated.

By transforming an appropriate host cell with the aforementioned expression vector, a transformant can be obtained. The host can be appropriately selected in relation with the vector, and for example, Escherichia coli, Bacillus subtilis, a bacterium of genus Bacillus, yeast, an insect, insect cells, animal cells and the like are used. As the animal cells, for example, HEK293T cells, CHO cells, COS cells, myeloma cells, HeLa cells, and Vero cells may be used. Transformation can be performed according to the known method such as a lipofection method, a calcium phosphate method, an electroporation method, a microinjection method, a particle gun method and the like, depending on a kind of the host.

By culturing the transformant according to the conventional method, an objective polypeptide is expressed.

For purifying a protein from the culture of the transformant, cultured cells are recovered, and suspended in an appropriate buffer, and the cells are destructed by a method such as ultrasound treatment, and freezing and thawing, and subjected to centrifugation or filtration to obtain a crude abstract. When the polypeptide is secreted into the culturing liquid, the supernatant is recovered.

Purification from the crude extract or the culturing supernatant can be performed by the known method or another equivalent method (e.g. salting out, dialysis method, ultrafiltration method, gel filtration method, SDS-PAGE method, ion exchange chromatography, affinity chromatography, reversed phase high performance liquid chromatography etc.).

(Kit)

A kit according to the present invention is a kit for cutting a target double-stranded nucleic acid, including the “set of two polypeptides exhibiting the nuclease activity” according to the present invention, nucleic acids encoding the set of polypeptides, or a vector including the nucleic acids, and a guide RNA including a sequence complementary to one sequence of a target double-stranded nucleic acid or a nucleic acid encoding it.

For example, the kit can be a kit including a total of 3 kinds of nucleic acids of nucleic acids encoding each of the set of two polypeptides exhibiting the nuclease activity, and a nucleic acid encoding the guide RNA, and in the kit, 3 kinds of nucleic acids may be introduced into 1, 2, or 3 vector(s). The guide RNA may be of two or more kinds.

A kit according to the present invention is a kit for cutting a target double-stranded nucleic acid, including the “set of two polypeptides exhibiting the nickase activity” according to the present invention, or nucleic acids encoding the set of polypeptides, or a vector including the nucleic acids, and a pair of guide RNAs including a sequence complementary to each sequence of the target double-stranded nucleic acid or nucleic acids encoding them.

For example, the kit can be a kit including 4 kinds of nucleic acids of nucleic acids encoding each of the set of two polypeptides exhibiting the nickase activity, and nucleic acids encoding a pair of guide RNAs, and in the kit, 4 kinds of nucleic acids may be inserted into 1, 2, 3 or 4 vector(s). The pair of the guide RNAs may be of two or more.

The kit according to the present invention can also be used in genome editing following cutting, and in that case, the kit may be provided with a reagent necessary for NHEJ or HDR.

A kit according to the present invention is a kit for suppressing expression of a target gene, including the “set of two polypeptides suppressing gene expression of a target gene” according to the present invention, or nucleic acids encoding the set of polypeptides, or a vector including the nucleic acids, and a guide RNA complementary to a partial sequence of a target gene or a nucleic acid encoding it.

For example, the kit can be a kit including a total of 3 kinds of nucleic acids of nucleic acids encoding each of the set of two polypeptides suppressing gene expression of a target gene, and a nucleic acid encoding a guide RNA, and in the kit, 3 kinds of nucleic acids may be inserted into 1, 2, or 3 vector(s). The guide RNA may be two or more kinds.

A kit according to the present invention is a kit for activating expression of a target gene, including the “set of two polypeptides activating gene expression of a target gene” according to the present invention, or nucleic acids encoding the set of polypeptides, or a vector including the nucleic acids, a guide RNA including a sequence complementary to a partial sequence of the target gene with an aptamer introduced therein or a nucleic acid encoding it, and an aptamer-binding protein ligated to a transcription activation domain or a nucleic acid encoding it.

For example, the kit can be a kit including a total of 4 kinds of nucleic acids of nucleic acids encoding each of a set of two polypeptides suppressing gene expression of a target gene, a nucleic acid encoding an aptamer and a guide RNA, as well as a nucleic acid encoding a transcription activation domain and an aptamer-binding protein, and in the kit, 4 kinds of nucleic acids may be introduced into 1, 2, 3, or 4 vector(s). The guide RNA may be of two or more kinds.

In the present invention, a set of two polypeptides activating gene expression of a target gene including a polypeptide, in which VP64 as a transcription activation domain is bound to the C-terminal side fragment of the Cpf1 protein; a nucleic acid encoding a guide RNA bound with a MS2-binding sequence, in which an aptamer-binding protein is MS2, and a transcription activation domain is p65 and HSF1; as well as nucleic acids encoding p65, HSF1 and MS2 are suitably used, and as a factor corresponding to VP64, MS2, p65 and HSF1, a transcription activation domain and an aptamer-binding protein such as those disclosed in Nature (2015) 517, 583-588 and nature protocols (2012) 7 (10), 1797-1807 can also be used.

As with the kit for activating expression of a target gene and the kit for suppressing expression of a target gene, the kit according to the present invention may be a kit for exerting the function based on a functional domain.

The kit may include the above-described “set of two nickase-active polypeptides” or the above-described “set of two nuclease-inactive polypeptides”.

The kit according to the present invention may be provided with other necessary reagents and instruments, and examples thereof are not limited to, but include various buffers, and a necessary primer, enzyme, manual and the like.

The disclosure of all patent documents and non-patent documents cited in the present specification are incorporated herein by reference as a whole.

Preparation of Plasmid Encoding Inducibly Associative Cpf1 Nuclease

cDNAs encoding an N-terminal side fragment and a C-terminal side fragment of Cpf1 derived from the Lachnospiraceae bacterium ND2006 in which codons had been optimized (LbCpf1) were prepared based on a plasmid (#69988) obtained from Addgene. cDNAs encoding drug switch proteins (FKBP, FRB) were prepared based on a human cDNA library. cDNAs encoding light switch proteins (pMag, nMagHigh1) were prepared according to a reference literature (Kawano, F. et al. Nat. Commun. 6, 6256 (2015)). During amplification of those dimerization domains (light switch proteins, drug switch proteins) by standard PCR, a linker composed of glycine and serine and a nuclear localization sequence were added to a 5′-terminus and a 3′-terminus of them. A construct of inducibly associative Cpf1 using the N-terminal side fragment and the C-terminal side fragment of Cpf1 and the dimerization domains was introduced into a pcDNA3.1 V5/His-A vector (by Invitrogen).

Preparation of Plasmid Encoding Divided dCpf1 Activator

To prepare a plasmid encoding a divided dCpf1 activator, dLbCpf1 was prepared by introducing E925A mutation into LbCpf1 to delete the nuclease activity by using standard overlap PCR. A cDNA encoding p65-HSF1 was obtained from an Addgene plasmid (#61423), and a linker composed of glycine and serine and a nuclear localization sequence were added to a 5′-terminus and a 3′-terminus of it by PCR. A construct of the divided dLbCpf1 activator composed of the N-terminal side fragment and the C-terminal side fragment of dLbCpf1 and p65-HSF1 was introduced into a pcDNA3.1 V5/His-A vector. To prepare SAM as a control, dCas9-VP64 and MS2-p65-HSF1 were amplified from Addgene plasmids (#61422 and 61423), and introduced into pcDNA3.1 V5/His-A.

Preparation of Plasmid Encoding crRNA and sgRNA

For expression of a crRNA in a mammalian cell using a human U6 promoter, a pSPgRNA vector (Addgene plasmid #47108) was used with modification. crRNAs targeting the Fluc reporter, DNMT1, GRIN2b, FANCF1, the GAL4-luciferase reporter, ASCL1, HBG1, IL1R2, IL1RN, and NGN3, respectively, with a stop codon introduced therein were prepared by introducing an oligo DNA into a BsmBI site of the modified pSPgRNA vector. An sgRNA into which an MS2-binding sequence had been introduced (called sgRNA 2.0) was amplified from an Addgene plasmid (#61424), and introduced into a pSPgRNA vector for use. sgRNAs targeting ASCL1, HBG1, IL1R2, IL1RN, and NGN3, respectively, were prepared by introducing an oligo DNA into a BbsI site of the sgRNA 2.0 vector.

Preparation of Reporter Plasmid

An Fluc reporter with a stop codon introduced therein was prepared by introducing firefly luciferase (Fluc) from a pGL4.31 vector (by Promega) into Hind III and Xho I sites of a pcDNA 3.1/V5-HisA vector, and introducing a stop codon and a PAM sequence by the Multi Site-Directed Mutagenesis Kit. A luciferase donor vector was prepared by introducing an inverted sequence of Fluc into Xho I and Hind III sites of a pColdI vector (by Clontech). A surrogate EGFP reporter was prepared by introducing mCherry and EGFP with a codon frame shift into Hind III and Xho I sites of a pcDNA 3.1/V5-HisA vector. In the preparation, a DNMT1 target sequence was introduced into EcoR I and BamH I sites between the mCherry and EGFP with a codon frame shift by using an oligo DNA.

Cell Culture

HEK293T cells (by ATCC) were cultured under the condition of 37° C. and 5% CO2 using Dulbecco's Modified Eagle Medium (DMEM, by Sigma Aldrich) to which 10% FBS (HyClone), 100 unit/mL penicillin, and 100 μg/mL streptomycin (GIBCO) had been added. HeLa cells (by ATCC) were cultured under the condition of 37° C. and 5% CO2 using Minimum Essential Media (MEM, Sigma Aldrich) to which 10% FBS, 100 unit/mL penicillin, and 100 μg/ml streptomycin had been added.

HDR Assay Using Luciferase Plasmid

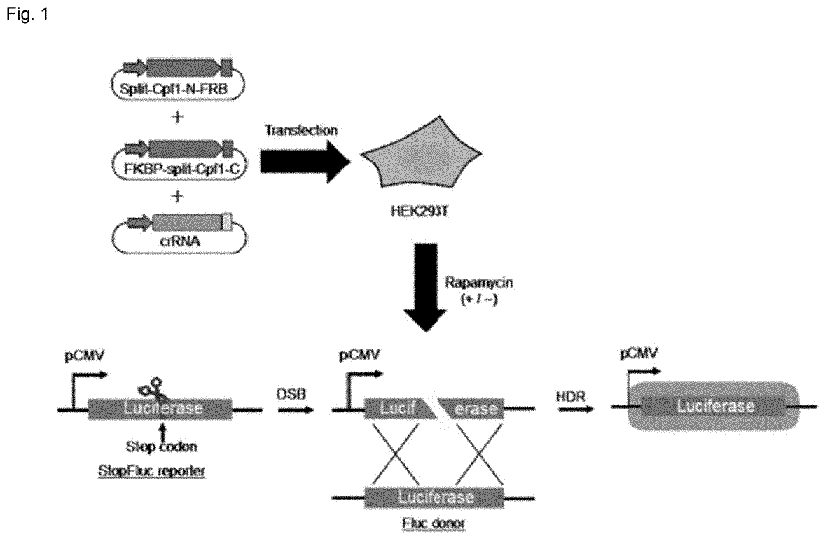

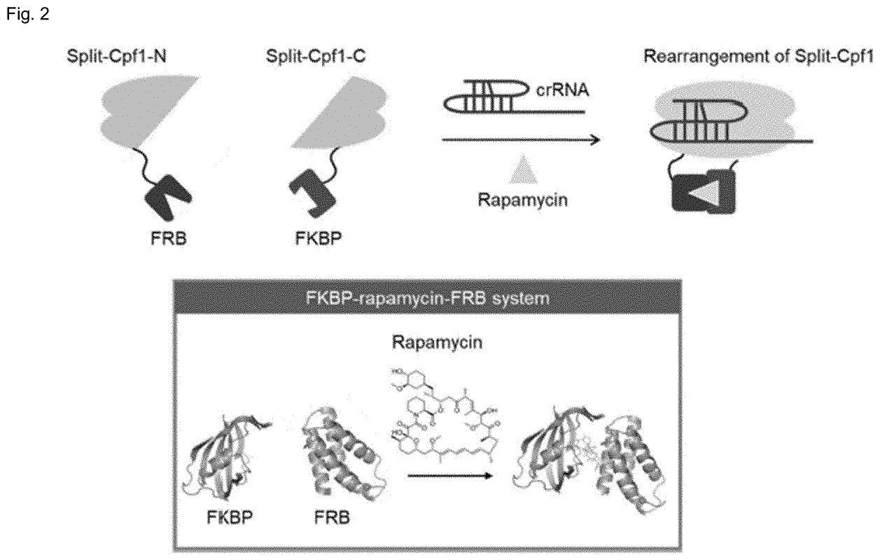

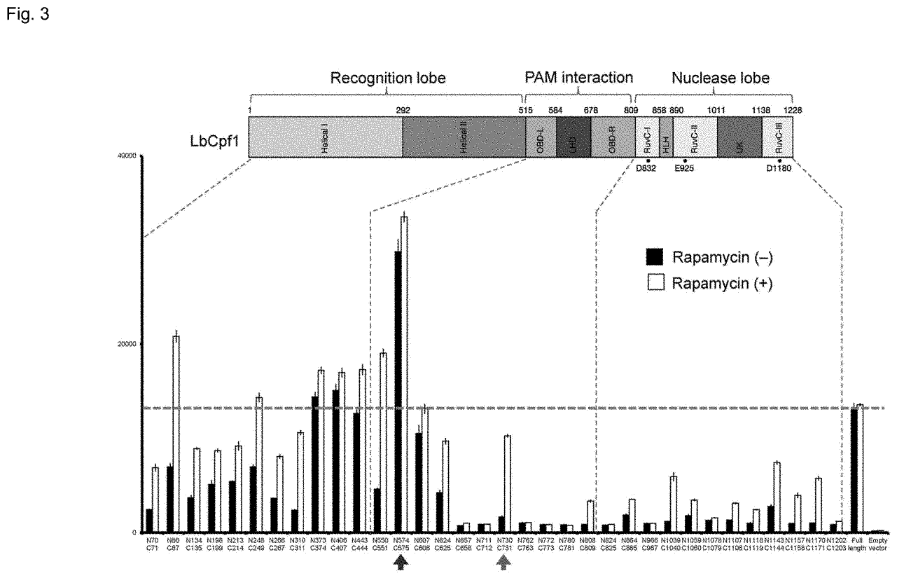

HEK293T cells were seeded on a 96-well black-walled plate (by Thermo Fisher Scientific) at the density of 2.0×104 cells/well, and cultured under the condition of 37° C. and 5% CO2 for 24 hours. Gene introduction into HEK293T cells was performed using Lipofectamine 3000 (by Thermo Scientific) according to a manual. The cells were transfected with plasmids encoding the N-terminal side fragment of LbCpf1 ligated with a dimerization domain, the C-terminal side fragment of LbCpf1 ligated with a dimerization domain, a crRNA, the Fluc reporter with a stop codon introduced therein, and the luciferase donor vector, respectively, at the ratio of 2.5:2.5:5:1:4. A total amount of the plasmids used for transfection is 0.1 μg/well. For evaluation of drug (rapamycin)-inducibly associative split-LbCpf1, the medium was replaced with 100 μL of DMEM containing 10 nM rapamycin 24 hours after transfection. For evaluation of light-inducibly associative split-LbCpf1, the sample was cultured under blue light irradiation, rather than use of rapamycin. For the blue light irradiation, a LED light source at 470 nm±20 nm (by CCS Inc.) was used. The light irradiation was performed with the intensity of blue light of 1 W/m2. After incubation for 48 hours, the medium was replaced with 100 μL of phenol red-free DMEM (by Sigma Aldrich) containing 500 μM D-luciferin (by Wako Pure Chemical Industries). After incubation for 30 minutes, luminescence measurement was performed with a plate reader (Centro XS3 LB 960, by Berthold Technologies) (FIG. 1, FIG. 2, FIG. 3).

Genome Editing for Inducibly Associative Type

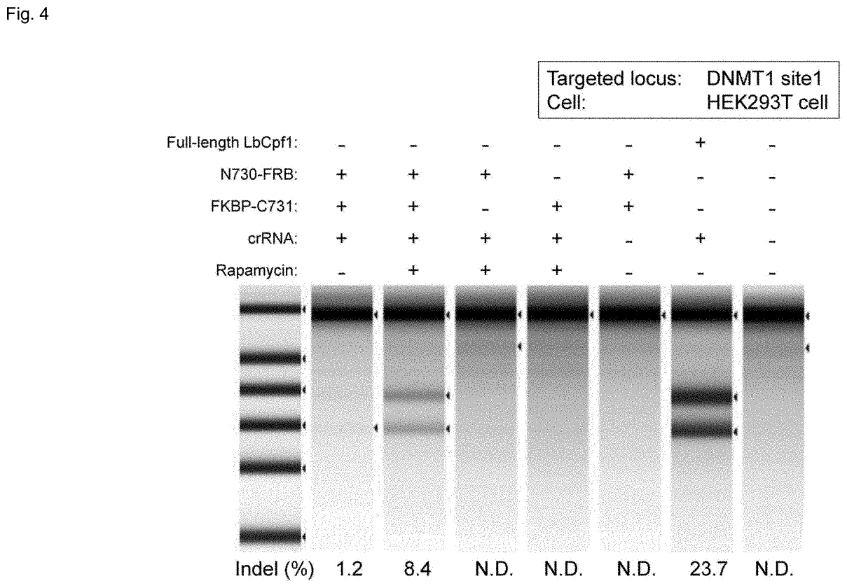

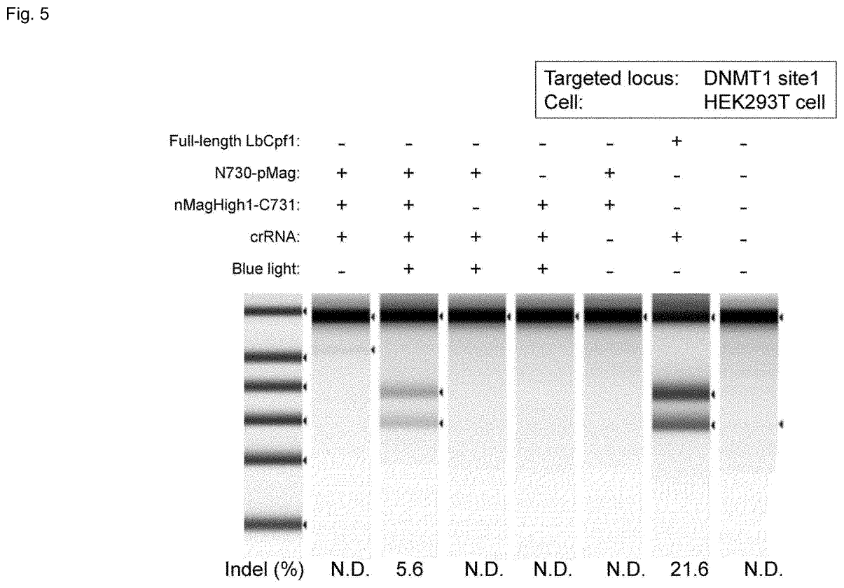

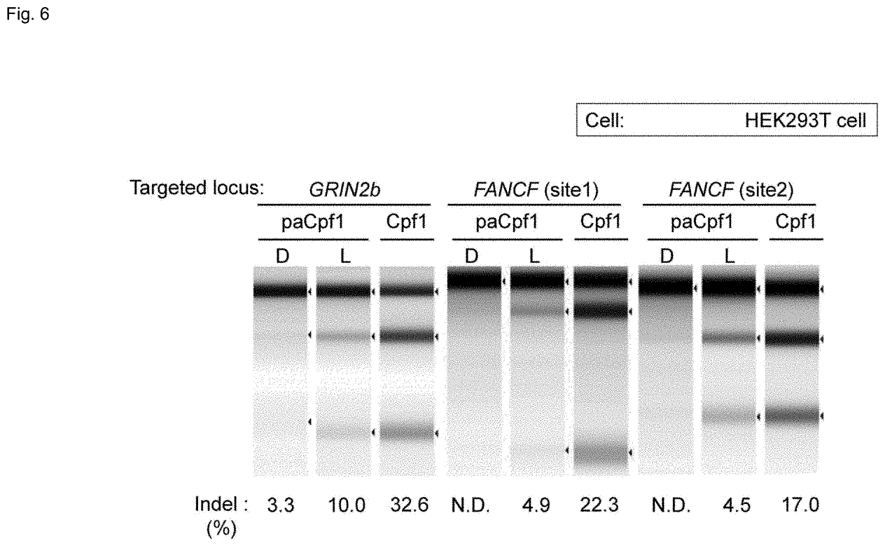

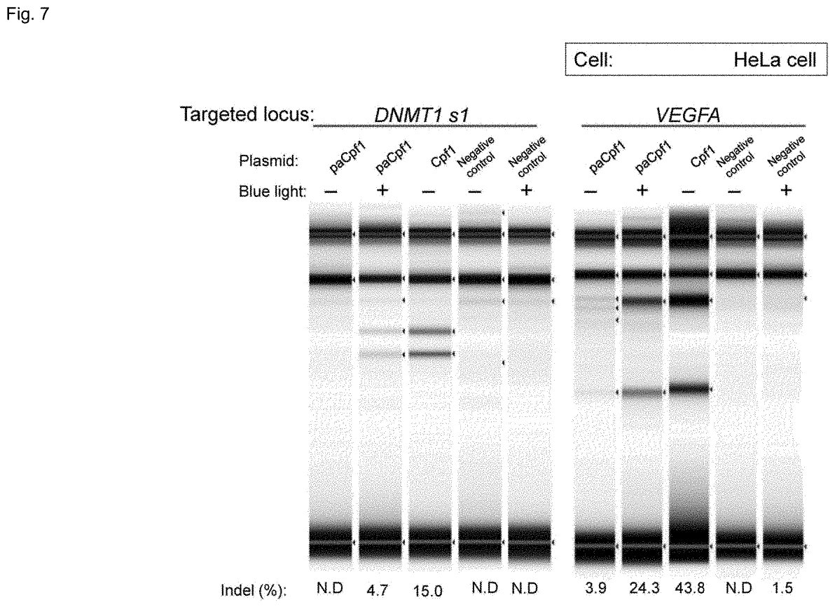

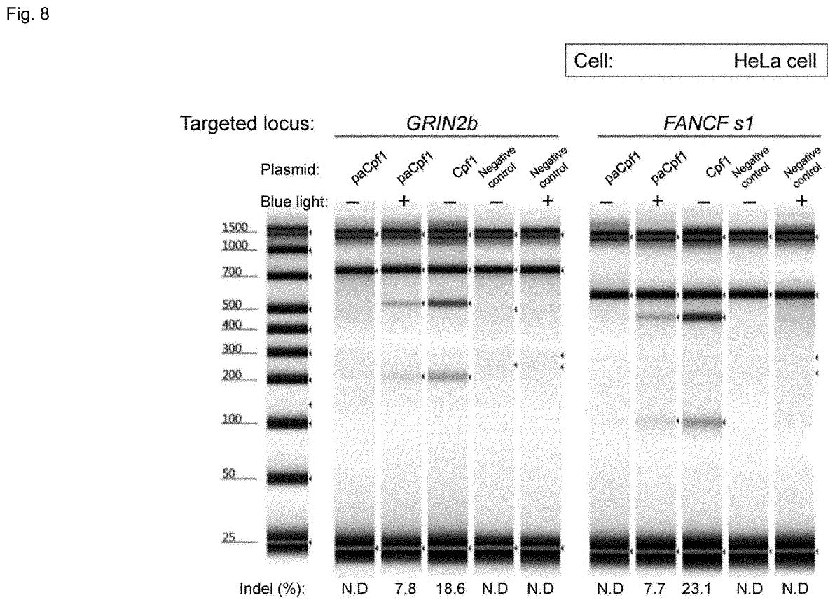

For evaluation of indel mutation by non-homologous end-joining (NHEJ), HEK293T cells were seeded on a 24-well black-walled plate (by Thermo Fisher Scientific) at the density of 1.0×105 cells/well, and cultured under the condition of 37° C. and 5% CO2 for 24 hours. Gene introduction into HEK293T cells was performed using Lipofectamine 3000 (by Thermo Scientific) according to a manual. The cells were transfected with plasmids encoding the N-terminal side fragment of LbCpf1 ligated with a dimerization domain, the C-terminal side fragment of LbCpf1 ligated with a dimerization domain, and a crRNA, respectively, at the ratio of 1:1:1. The cells were transfected with plasmids encoding full length LbCpf1 and a crRNA, as a positive control, at the ratio of 2:1. A total amount of the plasmids used for transfection is 0.5 μg/well. For HeLa cells, the cells were seeded on a 24-well black plate (by Thermo Fisher Scientific) at the density of 5.0×104 cells/well, and cultured under the condition of 37° C. and 5% CO2 for 24 hours. Gene introduction into HeLa cells was performed using X-tremeGENE 9 (by Sigma Aldrich) according to a manual. For evaluation of drug (rapamycin)-inducibly associative split-LbCpf1, the medium was replaced with DMEM containing 10 nM rapamycin 24 hours after transfection. For evaluation of light-inducibly associative split-LbCpf1, the sample was cultured under blue light irradiation, rather than use of rapamycin. Unless specified otherwise, after incubation for 24 hours, a genomic DNA was extracted by the Blood Cultured Cell Genomic DNA Extraction Mini Kit (by Favorgen) according to a manual (FIG. 4, FIG. 5, FIG. 6, FIG. 7, FIG. 8).

T7EI Assay for Quantitating Indel Mutation in Endogenous Gene

A genomic DNA containing a cutting site for split-LbCpf1 or full length LbCpf1 was PCR-amplified with the PrimeSTAR (R) HS DNA Polymerase (by TaKaRa). This PCR was performed under conditions for touchdown PCR as follows: 98° C., 3 min; (98° C., 10 sec; 72-62° C., −1° C./cycle, 30 sec; 72° C., 60 sec)×10 cycles; (98° C., 10 sec; 62° C., 30 sec; 72° C., 60 sec)×25 cycles, 72° C., 3 min. The amplicon obtained by PCR amplification was purified using the FastGene Gel/PCR Extraction Kits (by Nippon Genetics) according to a manual. The purified amplicon was mixed with 2 μL of the NEB buffer 2 (by New England Diolabs) for restriction enzymes and ultrapure water to reach 20 μL, and re-annealing was performed to form a hetero double-stranded DNA (95° C., 10 min; 90-15° C., −2.5° C./1 min). After performing re-annealing, the hetero double-stranded DNA was treated with the T7 endonuclease I (T7EI, by New England Biolabs) at 37° C. for 30 min, and analysis with a gel electrophoresis apparatus (Agilent 4200 TapeStation, by Agilent) was performed. Quantitation was performed based on the intensity of bands. Efficiency of indel mutation with split-LbCpf1 or full length LbCpf1 was calculated according to the following expression: 100×(1−(1−(b+c)/(a+b+c))½). Here, a indicates a PCR product which was not cut by T7EI, and b and c indicate PCR products which were cut by T7EI.

Evaluation of Spatial Genome Editing Using Surrogate EGFP Reporter

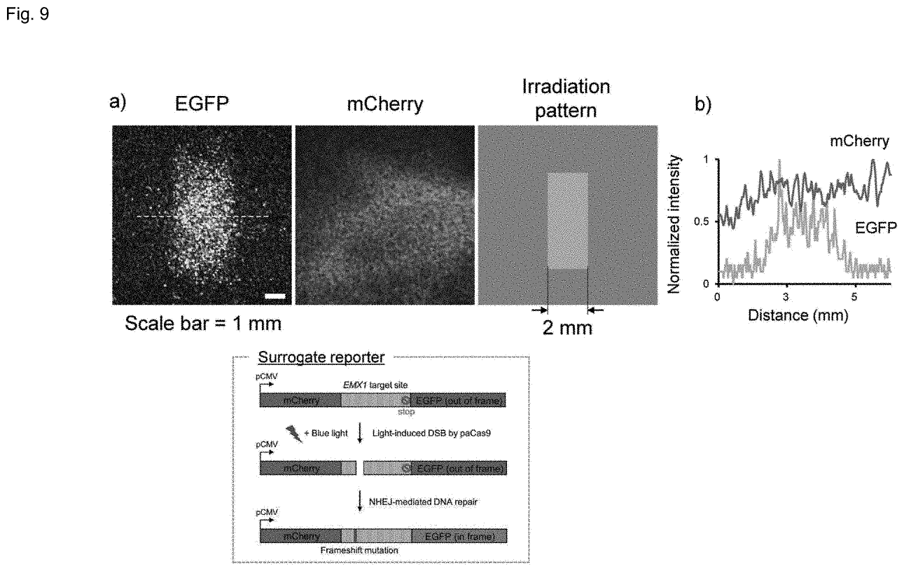

HEK293T cells were seeded on a 35 mm dish (by Iwaki Glass) surface-modified with fibronectin (by BD Biosciences) at the density of 8.0×105 cells/dish, and cultured under the condition of 37° C. and 5% CO2 for 24 hours. Gene introduction into HEK293T cells was performed using Lipofectamine 3000 (by Thermo Scientific) according to a manual. The cells were transfected with crRNAs targeting N730-pMag, nMagHigh1-C731, and DNMT1, and the surrogate EGFP reporter containing a target site for DNMT1 at the ratio of 1:1:2:6. A total amount of the plasmids used for transfection was 0.5 μg/dish. Twenty-four hours after transfection, irradiation was performed with blue light of a slit pattern of 2 mm using a photomask (24 hours, 37° C., 5% CO2). The cells were fixed by treatment with 4% paraformaldehyde for 15 min. An image was obtained with a stereomicroscope (M205 FA, by Leica), and subjected to image analysis with software (Metamorph, by Molecular Devices) (FIG. 9).

Evaluation of Transcription Activation Using GAL4-Luciferase Reporter

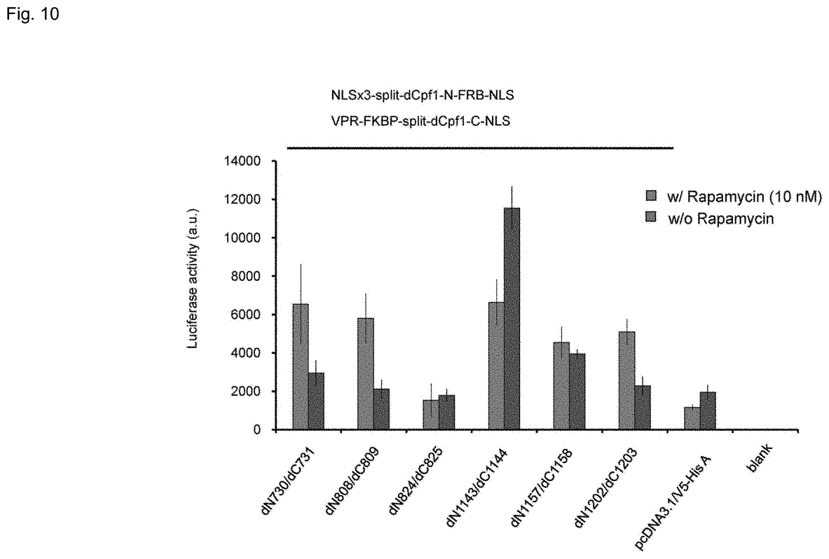

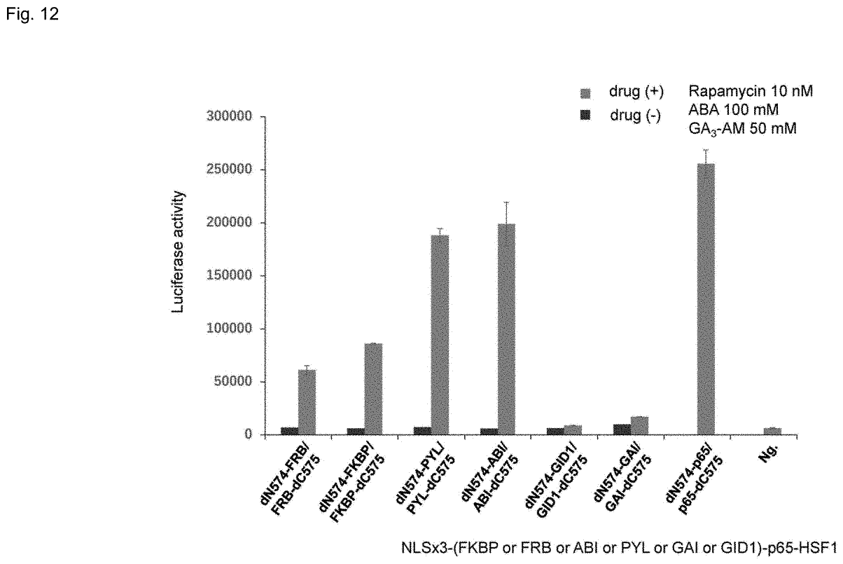

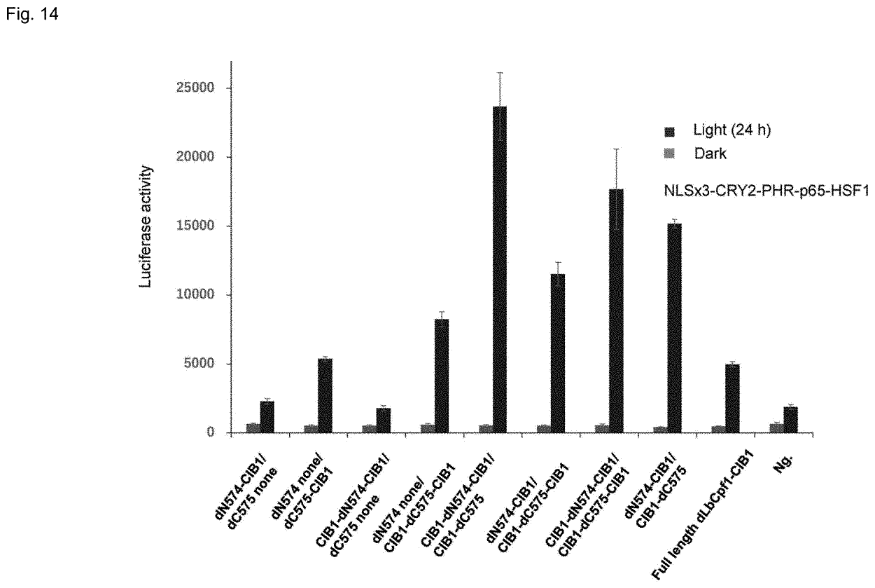

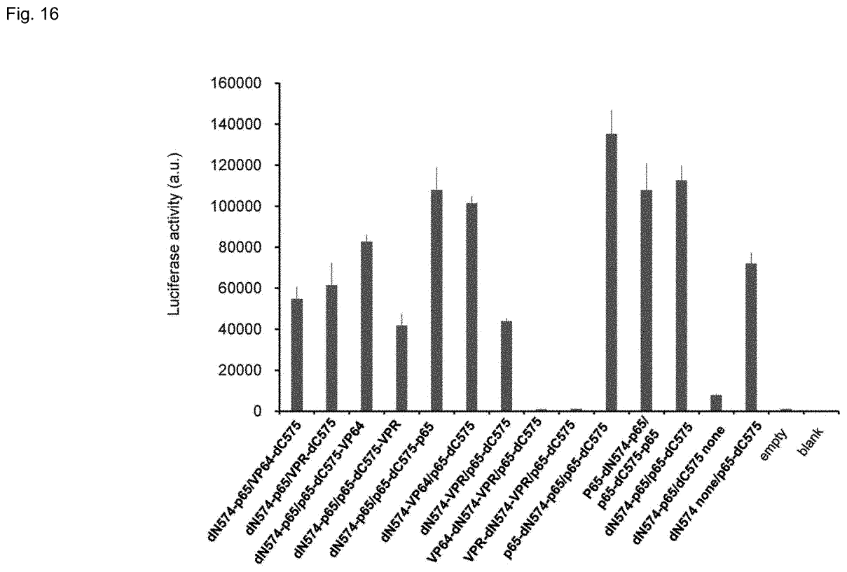

HEK293T cells were seeded on a 96-well black walled plate (by Greiner Bio-One) at the density of 2.0×104 cells/well, and cultured under the condition of 37° C. and 5% CO2 for 24 hours. Gene introduction into HEK293T cells was performed using Lipofectamine 3000 (by Thermo Scientific) according to a manual. The cells were transfected with the N-terminal side fragment of LbCpf1 ligated with a specific domain, the C-terminal side fragment of dLbCpf1 ligated with a specific domain, a crRNA, and the luciferase reporter at the ratio of 1:1:1:1. For the case with full length dLbCpf1 ligated with a transcription activation domain as a positive control, the cells were transfected with full length dLbCpf1 ligated with a transcription activation domain, a crRNA, and the luciferase reporter at the ratio of 2:1:1. A total amount of the plasmids used for transfection was 0.1 μg/well. Forty-eight hours after transfection, the medium was replaced with 100 μL of phenol red-free DMEM (by Sigma Aldrich) containing 500 μM D-luciferin (by Wako Pure Chemical Industries). Bioluminescence measurement was performed with a plate reader (Centro XS3 LB 960, by Berthold Technologies) (FIG. 10, FIG. 12, FIG. 14, FIG. 16).

HDR Assay for Spontaneously Associative Split-Cpf1

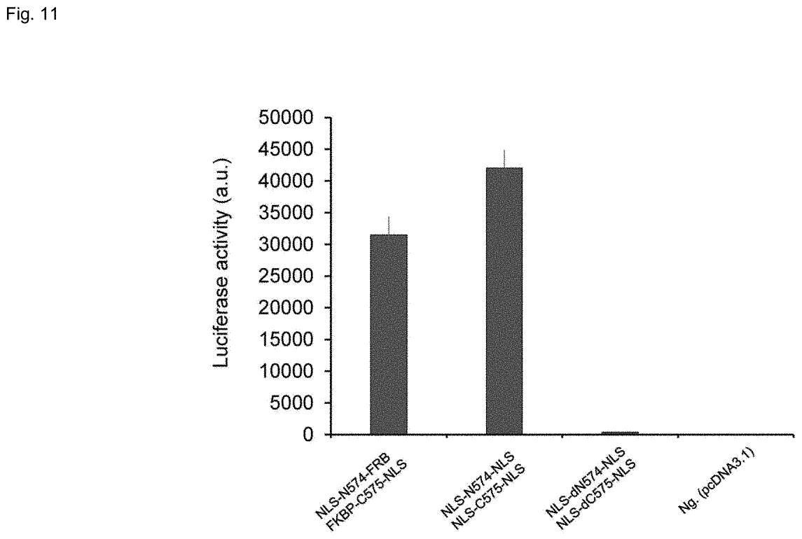

HEK293T cells were seeded on a 96-well black-walled plate (by Thermo Fisher Scientific) at the density of 2.0×104 cells/well, and cultured under the condition of 37° C. and 5% CO2 for 24 hours. Gene introduction into HEK293T cells was performed using Lipofectamine 3000 (by Thermo Scientific) according to a manual. The cells were transfected with plasmids encoding the N-terminal side fragment of LbCpf1 (N574), the C-terminal side fragment of LbCpf1 (C575), a crRNA, the Fluc reporter with a stop codon introduced therein, and the luciferase donor vector, respectively, at the ratio of 2.5:2.5:5:1:4. A total amount of the plasmids used for transfection was 0.1 μg/well. After incubation for 48 hours, the medium was replaced with 100 μL of phenol red-free DMEM (by Sigma Aldrich) containing 500 μM D-luciferin (by Wako Pure Chemical Industries). After incubation for 30 minutes, luminescence measurement was performed with a plate reader (Centro XS3 LB 960, by Berthold Technologies) (FIG. 11).

Quantitative Real Time PCR Analysis

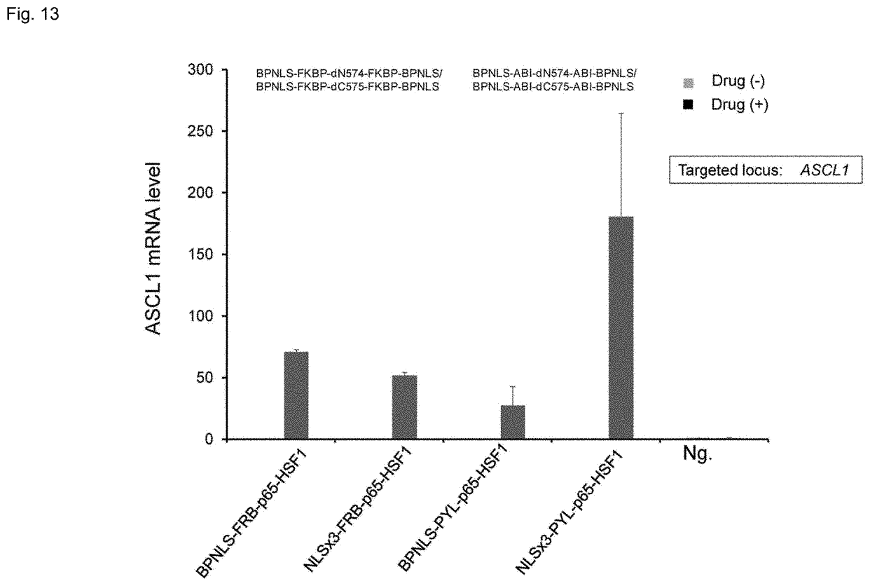

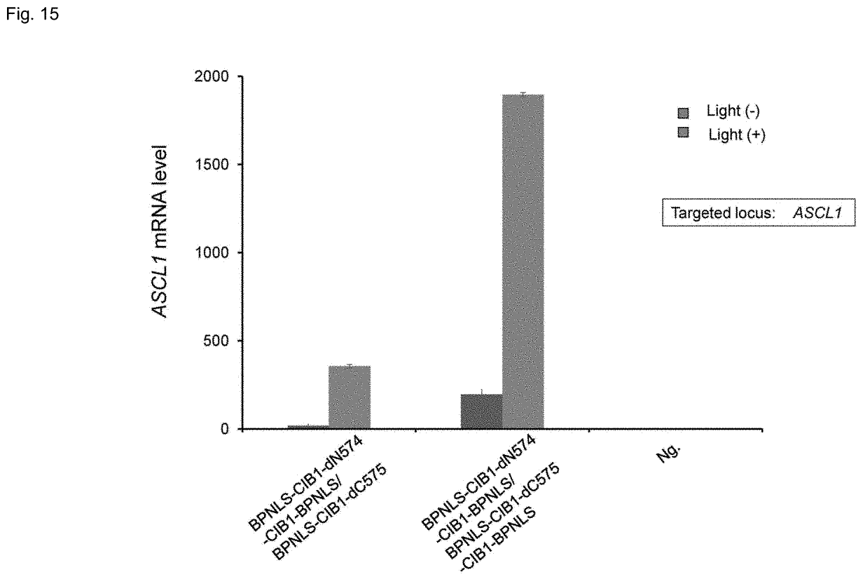

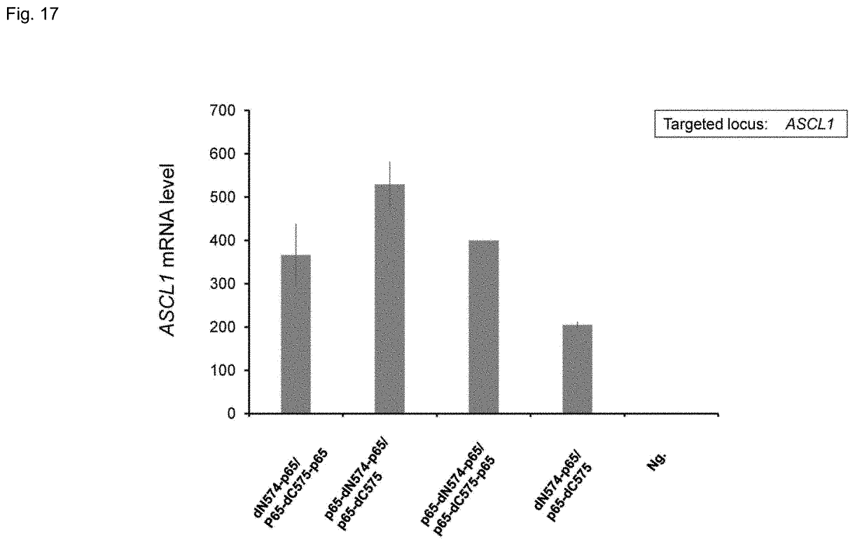

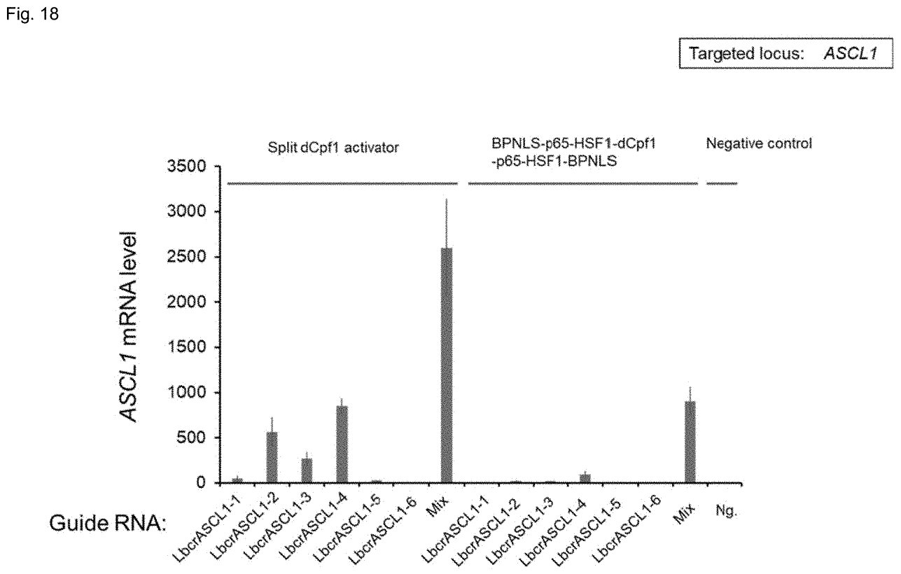

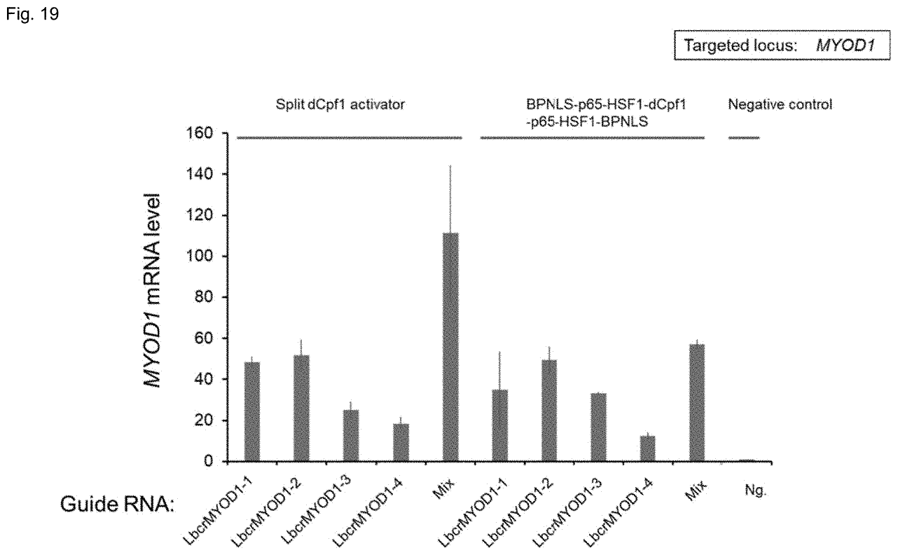

A total RNA was extracted using the Cells-to-Ct Kit (by Thermo Fisher Scientific) or the CellAmp Direct RNA Prep Kit (by TaKaRa) in combination with the PrimeScript RT Master Mix (by TaKaRa) and the SuperScript IV VILO Master Mix (by Thermo Fisher Scientific) according to a manual. Quantitative real time PCR analysis was performed using the StepOnePlus system (by Thermo Fisher Scientific) and the TaqMan Gene Expression Master Mix (by Thermo Fisher Scientific) according to a manual. TaqMan probes for detecting respective target genes and endogenous-controlled GAPDH (by Life technologies) were used. The TaqMan Gene Expression Assay ID is as follows: ASCL1: Hs04187546_g1, MYOD1: Hs02330075_g1, IL1RN: Hs00893626_m1, IL1R2: Hs01030384_m1, NGN3: Hs01875204_s1, HBG1: Hs00361131_g1, GAPDH: Hs99999905_m1). The relative mRNA level of each sample with respect to a negative control (obtained by treating cells with a vacant vector introduced therein in a dark place) was calculated by the standard ΔΔCt method (FIG. 13, FIG. 15, FIG. 17, FIG. 18, FIG. 19).

Culturing of iPS Cells, Transfection, Differentiation into Nerve Cells by Blue Light Irradiation

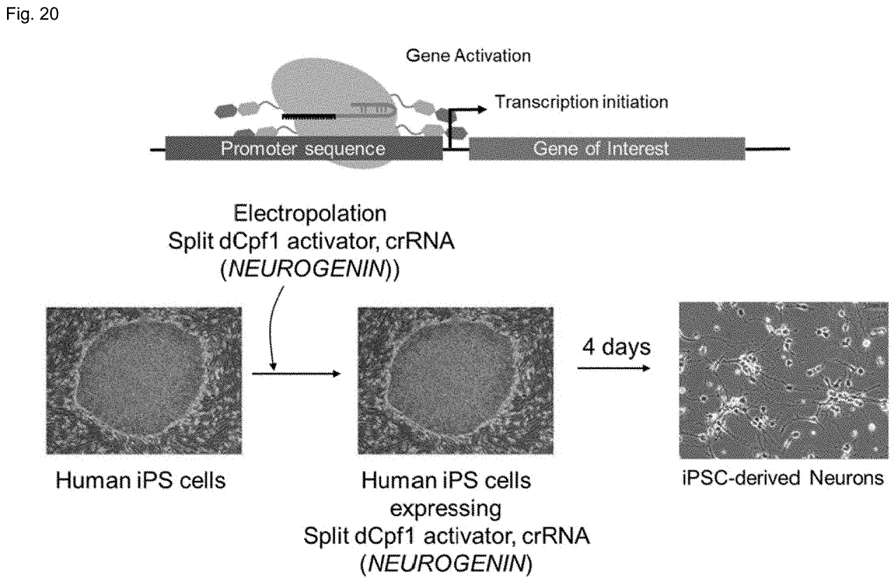

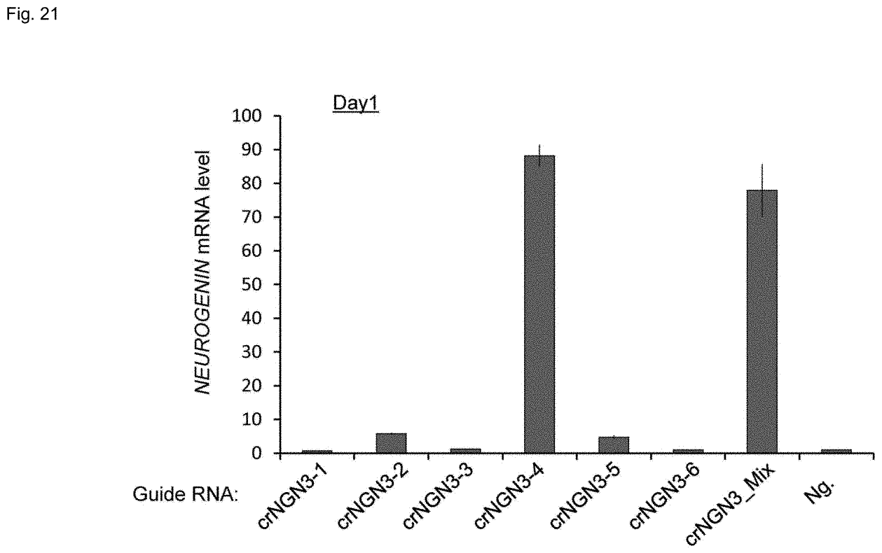

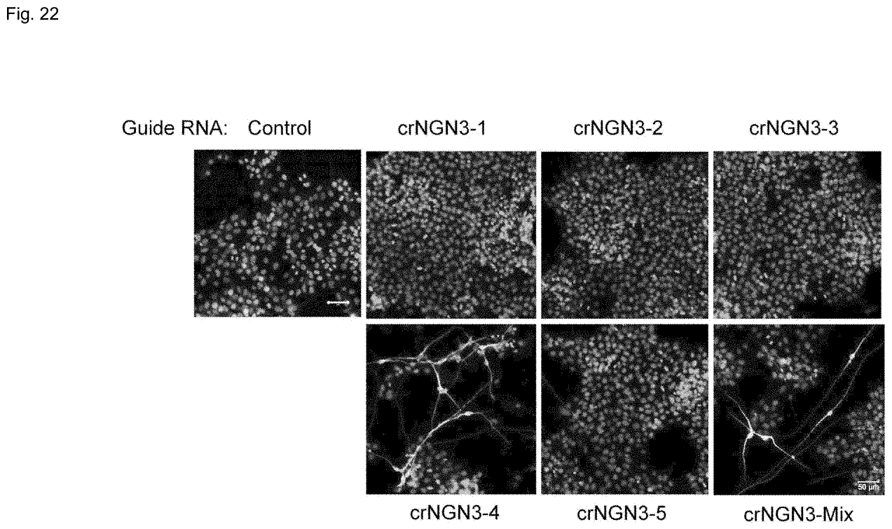

Human iPS cells (#454E2) were obtained from RIKEN Bio Resource Center, and cultured in an mTeSR1 medium (by Stemcell Technologies) using a 6-well culture plate (by Thermo Fisher Scientific) coated with Matrigel (by Corning, #354230). In order to introduce crRNAs targeting pCAG-BPNLS-p65-HSF1-NLS-dN574-p65-HSF1-BPNLS, pCAG-BPNLS-p65-HSF1-dC575-p65-HSF1-BPNLS, and NGN3 into 5.0×105 iPS cells, the 4D-Nucleofector (utilizing CA-137 program by Lonza) and the P3 Primary Cell 4D-Nucleofector X Kit S (by Lonza) were used. The transfected cells were seeded on an 8-well chamber slide (by Thermo Scientific) coated with Matrigel, at the density of 2.5×105 cells/well, and cultured with an mTeSR1 medium containing 10 μM ROCK inhibitor (by WAKO). A new mTeSR1 medium containing 10 μM ROCK inhibitor was added every day. Twenty-four hours after transfection, the sample was analyzed by quantitative real time PCR, and staining by a fluorescent antibody method was performed 96 hours after transfection (FIG. 20, FIG. 21, FIG. 22).

Analysis of Nerve Cells Resulting from Differentiation with Divided dLbCpf1 Activator by a Fluorescent Antibody Method

A sample was washed with PBS twice, and fixed with 4% paraformaldehyde (by WAKO) for 10 minutes, and thereafter, treated with PBS containing 0.2% Triton X-100 for 10 minutes. The sample was washed with PBS twice, blocked with 3% BSA and 10% FBS for 1 hour, and stained with the anti-beta III tubulin eFluor 660 conjugate (by eBioscience, catalog no. 5045-10, clone 2G10-TB3) for 3 hours. In addition, the anti-beta III tubulin eFluor 660 conjugate was used by diluting it with a blocking solution at 1:500. The sample was washed with PBS twice, and stained with the DAPI (by Thermo Scientific) for 10 minutes. The stained sample was fluorescently observed with a confocal laser scanning microscope (LSM710 by Carl Zeiss) mounted with an objection lens at magnification of 20.

Comparison Between Activation of Endogenous Gene by Divided dCpf1 Activator and that by dCas9-SAM

HEK293T cells were seeded on a 96-well plate (by Thermo Scientific) at the density of 2.0×104 cells/well, and cultured under the condition of 37° C. and 5% CO2 for 24 hours. Gene introduction into HEK293T cells was performed using Lipofectamine 3000 (by Thermo Scientific) according to a manual. A total amount of plasmids used for transfection was 0.1 μg/well. The cells were transfected with a cDNA encoding an N-terminal fragment of Cpf1 ligated with an activator domain (BPNLS-p65-HSF1-NLS-dN574-p65-HSF1-BPNLS) (the sequence is the same as SEQ ID No.: 15), a C-terminal fragment of dCpf1 ligated with an activator domain (BPNLS-p65-HSF1-dC575-p65-HSF1-BPNLS) (the sequence is the same as SEQ ID No.: 16), and a crRNA at the ratio of 1:1:1. For dCas9-SAM, the cells were transfected with a cDNA encoding dCas9-VP64, a cDNA encoding MCP-p65-HSF1, and sgRNA2.0 at the ratio of 1:1:1. Forty-eight hours after transfection, quantitative real time PCR (rtPCR) analysis was performed.

Gene Activation in Living Bodies of Mice (In Vivo)

Animal experiments were conducted in accordance with “Guidelines for Proper Conduct of Animal Experiments” by The University of Tokyo. In in vivo luciferase reporter experiment, plasmids incorporating a cDNA encoding the divided dCpf1 activator, the GAL4-UAS luciferase reporter, and a crRNA targeting the reporter or a crRNA targeting irrelevant human B4GALNT1 at the ratio of 1:1:1 were injected into 6-week-old female mice (BALB/c). For the injection, the TransIT-EE Hydrodynamic Delivery Solution (by Mirus Bio LLC) was used. The injection was performed with 0.1 mL of an injection solution per 1 g body weight of a mouse and a total amount of 75 μg of DNA per mouse. Twenty hours after injection, the skin of the abdominal part of each mouse was depilated with depilatory cream. Twenty-four hours after injection, bioluminescence imaging was performed using the Lumazone bioluminescence imager (NIPPON ROPER K.K.) and the Evolve 512 EMCCD camera (by Photometrics). Immediately before bioluminescence imaging, 200 μL of Hank's balanced salt solution containing 100 mM D-luciferin was injected into the abdominal cavity of each mouse, and bioluminescence images were obtained within 5 minutes after injection. For in vivo activation of an endogenous gene (ASCL1), a cDNA encoding the divided dCpf1 activator and a crRNA targeting ASCL1 or a negative control crRNA at the ratio of 1:1 were injected into each mouse with the TransIT-EE Hydrodynamic Delivery Solution. At that time, a total amount of 100 μg of DNA was used per mouse. Twenty-four hours after injection, the liver was taken out and put in the RNA1ater solution (by Invitrogen). This is for the purpose of preventing the decomposition of RNA. A total RNA was extracted from the liver with the Precellys Evolution tissue homogenizer (by Bertin Instruments) mounted with the Cryolys Evolution cooling system, the Precellys Lysing Kit CK28, and Nucleospin RNA, and a cDNA was synthesized using the Superscript IV VILO Master Mix. rtPCR was performed using the Luna Universal Probe qPCR Master Mix (by New England Biolabs), and analysis was performed with the StepOne Real-Time PCR System. TaqMan primers (by Life technologies) were used for detecting the ASCL1 gene and the endogenous-controlled GAPDH gene. TaqMan Gene Expression Assay ID is as follows: ASCL1: Mm03058063_m1, GAPDH: Mm99999915_g1. The relative mRNA level of each sample with respect to a negative control without transfection was calculated by the standard ΔΔCt method.

### SEQUENCE LISTING FREE TEXT

SEQ ID No.: 1 represents an amino acid sequence of the Vivid protein.

SEQ ID No.: 2 represents a full length amino acid sequence of LbCpf1.

SEQ ID No.: 3 represents an amino acid sequence of LpCpf1-NLS-3xHA tag.

SEQ ID No.: 4 represents an amino acid sequence of NLS-N730-FRB.

SEQ ID No.: 5 represents an amino acid sequence of FKBP-C731-NLS.

SEQ ID No.: 6 represents an amino acid sequence of NLS-N730-pMag.

SEQ ID No.: 7 represents an amino acid sequence of nMagHigh1-C731-NLS.

SEQ ID No.: 8 represents an amino acid sequence of NLSx3-dN730-FRB-NLS.

SEQ ID No.: 9 represents an amino acid sequence of VPR-FKBP-dC731-NLS.

SEQ ID No.: 10 represents an amino acid sequence of NLS-N574-NLS.

SEQ ID No.: 11 represents an amino acid sequence of NLS-C575-NLS.

SEQ ID No.: 12 represents an amino acid sequence of BPNLS-CIB1-dN574-CIB1-BPNLS.

SEQ ID No.: 13 represents an amino acid sequence of BPNLS-CIB1-dC575-NLS.

SEQ ID No.: 14 represents an amino acid sequence of NLSx3-CRY2-PHR-p65-HSF1.

SEQ ID No.: 15 represents an amino acid sequence of BPNLS-p65-HSF1-NLS-dN574-p65-HSF1-BPNLS.

SEQ ID No.: 16 represents an amino acid sequence of BPNLS-p65-HSF1-dC575-p65-HSF1-BPNLS.

1. A set of two polypeptides of a split Cpf1 protein, wherein the two polypeptides are a N-terminal side fragment of a Cpf1 protein and a C-terminal side fragment of the Cpf1 protein.

2. The set of polypeptides according to claim 1, wherein the set is a set of two fused polypeptides of a split Cpf1 protein in which one of the N-terminal side fragment and the C-terminal side fragment of the Cpf1 protein is bound to each of two polypeptides which form a dimer with dependence on light or in the presence of a drug.

3. The set of polypeptides according to claim 1, wherein the N-terminal side fragment of the Cpf1 protein and the C-terminal side fragment of the Cpf1 protein spontaneously associate.

4. The set of polypeptides according to claim 1, wherein the Cpf1 protein is nuclease-active.

5. The set of polypeptides according to claim 1, wherein the Cpf1 protein is nuclease-inactive.

6. The set of polypeptides according to claim 5, wherein a functional domain is bound to the N-terminal side fragment of the Cpf1 protein and/or the C-terminal side fragment of the Cpf1 protein.

7. The set of polypeptides according to claim 1,
wherein the Cpf1 protein is nuclease-inactive,
N-terminal side fragment of the Cpf1 protein and the C-terminal side fragment of the Cpf1 protein spontaneously associate, and
the N-terminal side fragment of the Cpf1 protein and/or the C-terminal side fragment of the Cpf1 protein is/are bound to one of two polypeptides which form a dimer with dependence on light or in the presence of a drug, and a functional domain is bound to the other of the two polypeptides which form a dimer with dependence on light or in the presence of a drug.

8. The set of polypeptides according to claim 1,
wherein the N-terminal side fragment of the Cpf1 protein and the C-terminal side fragment of the Cpf1 protein are any of the following:
any of combinations of two polypeptides obtained by cutting an amino acid sequence of SEQ ID No.: 2 at any position of position 69 to position 73, position 83 to position 89, position 131 to position 138, position 244 to position 252, position 265 to position 296, position 309 to position 312, position 371 to position 387, position 404 to position 409, position 437 to position 445, position 549 to position 552, position 567 to position 577, position 606 to position 609, position 619 to position 628, position 727 to position 736, position 802 to position 811, position 1037 to position 1042, position 1140 to position 1148, position 1155 to position 1161, and position 1163 to position 1178;
a combination such that a sequence of at least one fragment in any of the above combinations includes addition, substitution, or deletion of one to several amino acids; and
a combination such that a sequence of at least one fragment in any of the above combinations has 80% or more sequence identity with the above sequence.

9. A nucleic acid encoding the set of polypeptides according to claim 1.

10. An expression vector comprising the nucleic acid according to claim 9.

11. A method of cutting a target double-stranded nucleic acid, the method including:
a step of incubating the target double-stranded nucleic acid, the set of polypeptides according to claim 4.

12. A method of cutting a target double-stranded nucleic acid, the method including:
a step of incubating the target double-stranded nucleic acid, the set of polypeptides according to claim 4, and a pair of guide RNAs including a sequence complementary to each sequence of the target double-stranded nucleic acid, by irradiating light, or in the presence of a drug.

13. A method of suppressing or activating expression of a target gene, the method comprising:
a step of incubating the target gene and the set of polypeptides according to claim 6.

14. A method of suppressing or activating expression of a target gene, the method comprising:
a step of incubating the target gene, the set of polypeptides according to claim 6, a pair of guide RNAs including a sequence complementary to each sequence of the target double-stranded nucleic acid, by irradiating light, or in the presence of a drug.

15. A method of suppressing or activating expression of a target gene, the method comprising:
a step of incubating the target gene and the set of polypeptides according to claim 7, by irradiating light, or in the presence of a drug.


SEQUENCE LISTING (16 sequences)

SEQ ID NO: 1 - protein, 186 aa, NEUROSPORA CRASSA
MSHTVNSSTMNPWEVEAYQQYHYDPRTAPTANPLFFHTLYAPGGYDIMGYLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 2 - protein, 1239 aa, Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQ

SEQ ID NO: 3 - protein, 1273 aa, Artificial Sequence
  LpCpf1-NLS-3xHA tag
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGSYPYDVPDYAYPYDVPDYAYPYDVPDYA

SEQ ID NO: 4 - protein, 871 aa, Artificial Sequence
  NLS-N730-FRB
MGTPKKKRKVGGSGMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDEEFGGSGSSGGSGGSGGSGILWHEMWHEGLEEASRLYFGERNVKGMFEVLEPLHAMMERGPQTLKETSFNQAYGRDLMEAQEWCRKYMKSGNVKDLTQAWDLYYHVFRRISKGGSGGSGGGSGSGSGG

SEQ ID NO: 5 - protein, 642 aa, Artificial Sequence
  FKBP-C731-NLS
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGGSGSGSGGGTNNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGS

SEQ ID NO: 6 - protein, 922 aa, Artificial Sequence
  NLS-N730-pMag
MGTPKKKRKVGGSGMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDEEFGGSGSSGGSGHTLYAPGGYDIMGYLRQIRNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGGSGGSGGGSGSGSGG

SEQ ID NO: 7 - protein, 701 aa, Artificial Sequence
  nMagHigh1-C731-NLS
MGGSGSSGGSGGSGGSGHTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETEGGSGGSGGGSGSGSGGGTNNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGS

SEQ ID NO: 8 - protein, 894 aa, Artificial Sequence
  NLSx3-dN730-FRB-NLS
MGTASPKKKRKVEASASPKKKRKVEASASPKKKRKVEASGGSGMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDEEFGGSGSSGGSGGSGGSGILWHEMWHEGLEEASRLYFGERNVKGMFEVLEPLHAMMERGPQTLKETSFNQAYGRDLMEAQEWCRKYMKSGNVKDLTQAWDLYYHVFRRISKGGSPKKKRKV

SEQ ID NO: 9 - protein, 1222 aa, Artificial Sequence
  VPR-FKBP-dC731-NLS
MGSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFGGSGSSGGSGASGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGSGGSGGGSGSGSGGGTNNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGS

SEQ ID NO: 10 - protein, 597 aa, Artificial Sequence
  NLS-N574-NLS
MGTPKKKRKVGGSGMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVEFPKKKRKV

SEQ ID NO: 11 - protein, 686 aa, Artificial Sequence
  NLS-C575-NLS
MGPKKKRKVGGSGTNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGS

SEQ ID NO: 12 - protein, 1261 aa, Artificial Sequence
  BPNLS-CIB1-dN574-CIB1-BPNLS
MGKRTADGSEFESPKKKRKVGGSNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSGGSGGSGGSGGTMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVEFGGSGSSGGSGGSGGSGNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSGGSGKRTADGSEFESPKKKRKV

SEQ ID NO: 13 - protein, 1013 aa, Artificial Sequence
  BPNLS-CIB1-dC575-NLS
MGKRTADGSEFESPKKKRKVGGSNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSGGSGGSGGSGGTNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKKGS

SEQ ID NO: 14 - protein, 886 aa, Artificial Sequence
  NLSx3-CRY2-PHR-p65-HSF1
MASPKKKRKVEASASPKKKRKVEASASPKKKSKVEASGSGTGGSGSSGGSGGSGGSGMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAGGSGGSGGGSGSGSGGLEPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 15 - protein, 1276 aa, Artificial Sequence
  BPNLS-p65-HSF1-NLS-dN574-p65-HSF1-BPNLS
MGKRTADGSEFESPKKKRKVPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSGGSGGSGGSGGTPKKKRKVGGSGMSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVEFGGSGGSGGSGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSGSGKRTADGSEFESPKKKRKV

SEQ ID NO: 16 - protein, 1356 aa, Artificial Sequence
  BPNLS-p65-HSF1-dC575-p65-HSF1-BPNLS
MGKRTADGSEFESPKKKRKVPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSGGSGGSGGSGGTNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQEFGGSGGSGGSGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVSGSGKRTADGSEFESPKKKRKV